(19) United States
(12) Patent Application Publication
(10) Pub. No.: US 2022/0257643 A1
(43) Pub. Date: Aug. 18, 2022

(54) Long Chain Fatty Acid Compound Colloidal Sulfur Formulations Comprising Functional Ingredients For Acne Treatment And Other Application

(71) Applicant: Anna D Guanche, Calabasas, CA (US)

(72) Inventor: Anna D Guanche, Calabasas, CA (US)

(21) Appl. No.: 17/551,062

(22) Filed: Dec. 14, 2021

Related U.S. Application Data
(63) Continuation of application No. 63/199,217, filed on Dec. 14, 2020.

(30) Foreign Application Priority Data
Jan. 11, 2021 (US) 63199217

(51) Int. Cl. A61K 33/04; A61K 47/14; A61K 47/34; A61K 47/44; A61K 47/06; A61K 47/02; A61K 47/18

(52) U.S. Cl. CPC A61K 33/04; A61K 47/14; A61K 47/34; A61K 47/183; A61K 47/06; A61K 47/02; A61K 47/44

(57) ABSTRACT

Disclosed herein are compositions suitable for topical application to the skin of a mammal for the purpose of treating or preventing a skin condition or for daily use in the maintenance of healthy skin comprising colloidal sulfur as a primary or sole active pharmaceutical ingredient, present in the composition, typically in high concentrations (above 5%), along with one or more long chain fatty acids or long chain fatty acid compounds, and optionally additional functional ingredients, such as squalane, silicone(s), or both, the compositions capable of providing detectable or significant anti-fungal activity, anti-yeast activity, anti-bacterial activity, anti-pruritic activity, and anti-inflammatory activity. In some respects, the compositions described herein are capable of providing one, some or each of detectable or significant anti-fungal activity, anti-yeast activity, anti-bacterial activity, anti-pruritic activity, and anti-inflammatory activity.

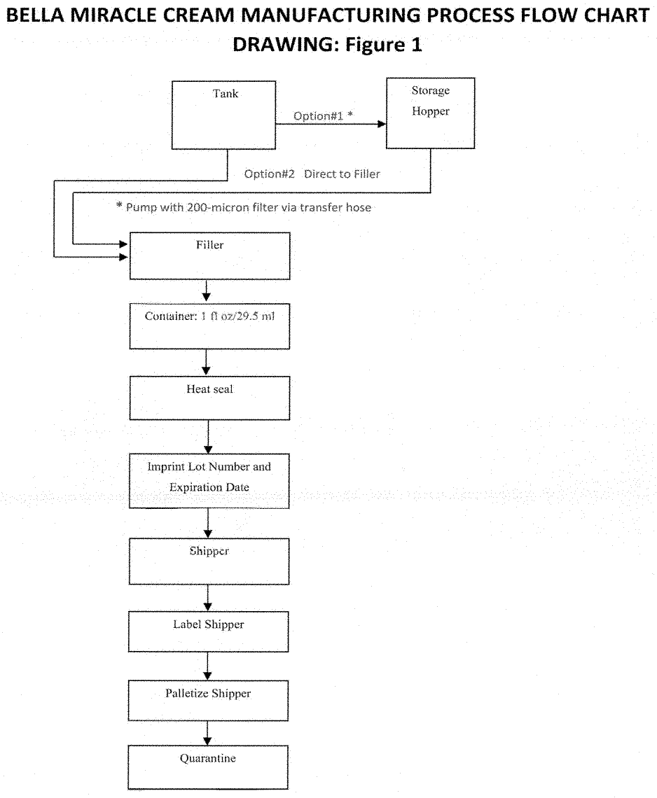

### BACKGROUND OF THE INVENTION

For an effective acne treatment, it has been said that the people of Rome bathed in warm mineral and sulfur-infused baths. Indeed, the use of sulfur in skincare reportedly dates back to the time of Hippocrates himself. In fact, sulfur as a treatment for cutaneous disease, was described in the Ebers Papyrus, an ancient Egyptian scroll, dated circa 1550 BCE.

There are several forms of sulfur products that have been applied to the skin since such ancient practices, such as precipitated sulfur. One such product is colloidal sulfur. Colloidal sulfur (i.e., sulfur colloidale) is a fine sulfur powder prepared by repeated precipitation, typically first from polysulfides with protein and then from a slightly alkaline solution with ethanol or acetone. It typically has a greyish-white color. Colloidal solutions in water typically are milky, and bluish when viewed against the light.

Colloidal sulfur was allegedly first prepared in 1888 by Debus. Sven Odén1a in 1911 made further extensive investigations of this product. Sulphur was frequently used for local application to the skin many years before dermatology became a recognized specialty. Colloidal sulfur, however, was not widely used as of the 1930s. In the 1930s, colloidal sulfur began to receive some attention as a product for treatment of skin conditions, such as acne. U.S. Pat. No. 1,917,351, published in 1933, for example, describes colloidal hydrophilic sulfur formulations for acne treatment. In 1935, Hirma E. Miller published an article highlighting the use of colloidal sulfur in dermatology. Arch Derm Syphilol. 1935;31(4):516-525. doi:10.1001/archderm.1935.01460220074008.

Sulfur products became widespread anti-acne treatments in the middle of the 20^{th} century, and sulfur today is an FDA-approved ingredient that is commonly found in over-the-counter products for acne. 5% sulfur is reportedly the most common strength in such products and most products that include sulfur today include sulfur in combination with benzoyl peroxide, salicylic acid, resorcinol, sodium sulfacetamide, or sodium sulfacetamide.

Some preparations/formulations containing sulfur can cause some skin discoloration, dry skin, or irritation, or can have a displeasing odor. Consequently, by the early 2000s sulfur medications were “used less often in patients with acne.” John J. Russel, Topical Therapy for Acne, Am Fam Physician. 2000 Jan 15;61(2):357-365 (see also Gupta A K, Nicol K. The use of sulfur in dermatology. J Drugs Dermatol. 2004 July-August;3(4):427-31. PMID: 15303787, similarly reporting more extensive prior use). Attempts to address such shortcomings associated with sulfur formulations/preparations led to combination formulations, such as those described above. For example, Medicis Pharmaceuticals developed formulations containing sulfur, sodium sulfacetamide and a pharmaceutically acceptable carrier (see U.S. Pat. No. 6,905,675). Such products are sold under the brand PLEXION™.

It has been recently reported in review of cosmetic products that different formulations of sulfur can, however, also overcome some of the odors and drying effects of sulfur formulations. Anna Hunter, Get the Gloss, Apr. 10, 2018, https://www.getthegloss.com/article/the-best-sulphur-products-for-acne. However, such positive reports are coupled with the continued warning that such products “could actually throw the skin's natural balance out of whack, which is bad news for breakout . . . such that [sulfur] should be seen as a last resort [product] . . . ” Clearly, given the significant effort and more than a century of experience in developing sulfur formulations for the treatment of acne, developing formulations that are effective while overcoming some or all of these issues will require application of inventive ingenuity. Such formulations and methods of using the same are provided by the invention described in this disclosure.

### PRINCIPLES OF CONSTRUCTION & ABBREVIATIONS

The following principles are applicable to interpreting the disclosure provided here. These principles apply unless clearly contradicted by express statement, context, or plausibility.

For sake of conciseness, well-known symbols are often used herein. For example, (“e.g.,”) “&” is used for “and” & “˜” is used for “about.” The symbols >, ≥, ≤ and < are given their ordinary meaning (e.g., >1 means “greater than 1”, <2 means “less than 2,” e.g., >2 Xs means “more than two Xs,” “≤” means “less than or equal to” and “≥” means “equal to or more than”) A slash “/” can indicate “or” (A/B means A or B) or can indicate an element with 2 names.

Terms in the singular implicitly convey corresponding disclosure of the plural & vice versa (e.g., a passage referring to use of an element implicitly discloses the corresponding use a plurality of such elements). The terms “a,” “an,” “the,” & similar referents likewise refer to both the singular & the plural of the associated element (e.g., “a X” means “≥1 Xs”).

The inclusion of “(s)” after an element indicates that ≥1 of such an element is present, performed, & the like. E.g., a composition comprising “X(s)” comprising “Y(s)” means a composition including one or more Xs collectively or individually comprising one or more Ys.

The abbreviation “WRT” means “with respect to.” “ACB” in connection with steps/elements means “are characterized by” (e.g., methods ACB steps) & “CB” is used to abbreviate the phrase “characterized by.” “AW” means “associated with.” “ACA” means “are characterized/characterizable as” & ICA means “is characterized/characterizable as.”

Terms such as “here” & “herein” means “in this disclosure.” The abbreviation “TD” similarly means “this disclosure.” The term “i.a.” (sometimes “ia” or “ia”) means “inter alia” or “among other things.” “Also known as” is abbreviated “aka.” “ORT” means “otherwise referred to.” The modifier “OTI” means “of the invention.” “AOTI” means “aspect(s) of the invention.” “ATAOTI” means “adaptable to” AOTI(s). “PMCs” means “principles, methods, or compositions.” “ITA” means “in the art.” “KITA” means “known in the art.” “POOSITA” means “person of ordinary skill in the art.” “DEH” means “discussed elsewhere here.” “DFEH” means “discussed further elsewhere herein.” “CEH” means “cited elsewhere herein.” “EH” means “elsewhere herein.” “SFE” means “see, for example.”

In the absence of other definition or understanding ITA, the term “some” WRT elements of a method or composition means “2 or more” & WRT a part of a whole means “at least 5%” (i.e., ≥5%). The abbreviation OSMGAOA means “one, some, most, generally all (i.e., at least 75%), or all,” each of which is an independent aspect of the described feature.

Fragments of abbreviations described can be used in the disclosure. E.g., “OSMOA” means “one, some, most or all” &“GAOA” means “generally all or all.” Abbreviations also can be combined, e.g., “WRT OSMOA” means “with respect to one, some, or all.”

The modifier “DOS” means detectable or significant/detectably or significantly. “Significant” means results that are statistically significant using an appropriate test in the given context (e.g., p≤0.05/0.01).

Lists of elements can be employed for conciseness. Unless indicated, each member of each list is an independent AOTI.

Ranges of values are used to concisely refer to each value in the range and within an order of magnitude of the range endpoints without having to explicitly write each value. E.g., a recited range of 1-2 implicitly discloses each of 1.0, 1.1, 1.2, 1.3, . . . 1.8, 1.9, & 2.0 & a recited range of 10-20 is to be interpreted as implicitly providing support for each of 10, 11, 12, 13, . . . 19, & 20). All recited ranges include the end points of the provided range, regardless of how the range is described, unless the exclusion of such endpoints is clearly indicated, regardless of the terminology used to describe the range. E.g., a range “between 1 & 5” will include 1 & 5 in addition to 2, 3, & 4 (& all numbers between such number within an order of magnitude of such endpoints & within such endpoints, in this example 1.1 & 4.9).

Terms of approximation, e.g., “about” (“˜”) or “approximately” are used to conveniently refer to a range of closely related values or where a precise value is difficult to measure or measurement difficult to define. The scope of value modified by a term of approximation will depend on the context of the disclosure or understanding of POOSITA. Absent such guidance, terms such as “about” (“˜”) should be understood as meaning +/−10% of the indicated value(s). All exact values provided herein are representative of corresponding approximate values & vice versa (e.g., all exact exemplary values provided WRT a particular factor or measurement can be considered to also provide a corresponding approximate measurement, modified by “about” or “˜,” e.g., disclosure of “˜10” implicitly discloses both 10 exactly & 9-11). Ranges described with one or more approximate numbers should be interpreted as indicating that all endpoints & other relevant values encompassed by the range may be similarly described, regardless of any different presentations included in TD (e.g., “˜10-20” should be interpreted in the same manner as “˜10-˜20”).

Use of the term “or” herein is not meant to imply that alternatives are mutually exclusive. In other words, “or” means “and/or.” The occasional explicit use of “and/or” herein has no effect on this interpretation of “or.” The scope of “or” meaning “and/or” in a phrase such as “A, B, and/or C” implicitly supports each of: A, B, & C; A, B, or C; A or C; A or B; B or C; A & C; A & B; B & C; A (alone); B (alone); & C (alone).

The term “also” means “also or alternatively” (“AOA”).

Any reference to “combination” or “combinations” of listed elements means combination(s) of any or all thereof. The abbreviation “CT” means “combination(s) thereof.” E.g., the statement “A, B, C or CT” means A; B; C; A and B; A and C; B and C; or A, B, and C.

No claim here is meant to be interpreted under “means-plus-function” construction unless such intent is clearly indicated by use of the terms “means for” or “step for.” Terms such as “configured to” or “adapted to” are not intended to suggest a “means-plus-function” interpretation, but, rather, indicate an element is configured to, designed to, selected to, or adapted to achieve a certain performance, characteristic, property, or the like using the principles described herein or that are generally KITA.

“Appreciably comprises” (“AC”) means ≥1% of a composition, component, or collection is composed of a referenced element. The phrase “appreciably associated” means ≥1% of an element is AW another element.

“Materially comprises” (“MC)” means ≥5% of the composition, component, or collection is/is made of the subject element. Phrases such as “materially associated” and “in material part” should be similarly interpreted.

“Considerably comprises” (“CC”) means that ≥20% of a composition, component, or collection is/is made of referenced element. Phrases such as “a considerable amount” have a corresponding meaning.

“Predominately comprises” (“PC”) means “most,” i.e., more than ½ (i.e., >50%) of a feature (e.g., a composition or a population).

Amounts of compositions are provided on a weight percent (weight/weight) basis, on a molecule/molecule basis, or other relevant basis used in the context of the relevant disclosure. E.g., if a composition is described as “predominately comprising” (or “predominately comprises”) (“PC”) element/species A, more than 50% of the composition on a molecular or weight basis will be made up of element/species A). Wherever this term is used it should be understood as simultaneously providing support for ≥60%, ≥70%, & ≥80% of the component or composition or collection being made up of the feature, species, or element at issue.

“Generally consists of” (“GCO) and similar phrases such as “generally is,” “generally are,” “generally most,” “generally all,” “generally,” or “generally is composed of” means that ≥75% of the composition, collection, or component is or is made up of the referenced element.

“Substantially consists of” (“SCO”) means ≥90% of the referenced composition, collection, or component is made up of the referenced element. “Substantially associated” means that ≥90% of a referenced item is AW a second item. Phrases like “substantially all,” “nearly all,” & “nearly entirely,” should be similarly construed.

Phrases such as “consists essentially of” (“CEO”) or “essentially” WRT to a step/element means that any step/element that maintains the fundamental features of the referenced element/step or retains the desired function of such element/step. The fundamental features & functions of elements and steps will be clear to PHOSITA from the disclosure in view of the art. Where features & functions are not clear, any such phrase should be interpreted as meaning “substantially consists of.”

Elements that “consist of” a step/element are limited to the step or element, within bounds of detection and as practically construed ITA.

The terms “including”, “containing”, & “having” should be interpreted openly herein, e.g., as meaning “including, but not limited to”, “including, without limitation”, or “comprising”, unless otherwise such a meaning is clearly contradicted. Terms such as “comprising”, “having”, “including”, or “containing” should be similarly construed when applied to the performance of a step, e.g., comprising means including any detectable amount of an element or including any detectable performance of a step,

A description of any AOTI using terms such as “comprising” or similar term (e.g., “including”) WRT a step/element simultaneously implicitly discloses corresponding AOTIs that (1) appreciably comprise (“AC”) the step/element, (2) materially comprise (“MC”) the step/element, (3) considerably comprise (“CC”) the step/element, (4) predominately comprise (“PC”) (ORT as “mostly” or “primarily” comprises) the step/element, (5) generally consists of (“GCO”) the step/element (or is “generally adapted” to, is “generally composed” of, “generally is,” “generally only” is/are, “generally are,” the element, & the like), or (6) substantially consists of (“SCO”) (or “substantially is” or “substantially only” is/are) the step/element, (7) consists essentially of (CEO) the element/step, or (8) consists of (“CO”) of the step/element (or “only is” or “only contains”). E.g., disclosure of a composition comprising “element X” correspondingly implicitly discloses compositions that appreciably, materially, considerably, or predominately comprise (i.e., ACMCCCPC), and generally, substantially, essentially, or only/completely consist of (GCOSCOCEO or CO) element X. In such lists a lower case “o” means “or.” E.g., a composition that “PCGCOSCOoCO element X” is a composition that primarily comprises, generally consists of, substantially consists of, or consists of element X.

Changes to tense or presentation of phrases defined herein (e.g., using “comprises predominately” instead of “predominately comprises”) do not modify the meaning of the defined phrase.

Unless otherwise indicated, compositions specifying a percentage are by weight unless a different value would be understood ITA. If a variable is not accompanied by a value, any previously provided value typically applies.

As used herein, the term “formulation” or “formulations” can be used synonymously with “composition” or “compositions”. For example, an aspect describing one or more characteristics of a composition of the invention also describes one or more characteristics of the invention when the invention is referred to as a formulation. Both terms are used herein to refer to the combination of ingredients providing the characteristics of a final product (e.g., in terms of efficacy, stability, and/or side effects, etc.) described herein.

All references, including publications, patent applications, & patents, cited herein, including patents & patent applications cited above, are hereby incorporated by reference to the same extent as if each reference were individually & specifically indicated to be incorporated by reference & were set forth in its entirety herein. Accordingly, the reader should review & consider such references in understanding the full content of TD. E.g., unless clearly contradicted by context or explicit statement, the disclosure of such documents relating to formulations, methods of production, & methods of use of compositions & devices can be combined with the teachings provided herein to provide additional useful compositions & applications. However, the reader should understand that the citation & incorporation of patent documents herein is limited to the technical disclosure of such patent documents & does not reflect any view of the validity, patentability, or enforceability of such patent documents. Moreover, in the event of any conflict between TD & the teachings of such documents, the content of TD will control WRT properly understanding the various aspects OTI. Numerous references have been included in TD to incorporate information available from other sources that illustrate the scope OTI or aid in putting aspects of it into practice. While efforts have been made to include the most relevant references for such purposes, readers will understand that not every aspect of every cited reference will be applicable to the practice OTI.

All headings & sub-headings (e.g., “Principles of Construction”) are used for convenience only & do not limit the scope of any AOTI. Aspects OTI described under a heading can apply or combine with other AOTI.

Numerous examples & aspects are provided here to exemplify and clarify the invention. No example, aspect, or combination or pattern thereof is intended to pose a limitation on the scope OTI. In general, AOTI should not be limited to any particular provided exemplary processes, compositions, or methodologies. All methods described herein can be performed in any suitable order unless otherwise indicated herein or otherwise clearly contradicted by context or plausibility. Unless clearly indicated or contradicted by context or plausibility, the elements of a described composition, device, or method can be combined in any suitable manner & by any suitable method and any combination of the various elements, steps, components, or features or variations thereof are AOTI.

No part of this specification should be construed as indicating any element or step is essential to the practice OTI unless as much is explicitly stated. Unless expressly otherwise indicated, description of terms KITA is for exemplifying versions or embodiments only & not intended to limit the scope of any aspect OTI. Unless defined otherwise, technical & scientific terms used herein have the same meanings as commonly understood by POOSITA & implicitly comprise the broadest interpretation based on such usage as well as any narrower interpretation(s) based on specific descriptions provided. In general, any methods & materials similar or equivalent to those described can be used in the practice or testing of embodiments OTI, methods, devices, & materials DH.

### SUMMARY OF THE INVENTION

Disclosed herein are compositions suitable for topical application to the skin of a mammal, such as a human, for the purpose of treating or preventing a skin condition, or for daily use in the maintenance of healthy skin, comprising sulfur as a single active ingredient, present in the composition along with one or more long chain fatty acids or long chain fatty acid compounds (e.g., long chain fatty acid alcohols, long chain fatty acid derivatives, and the like). In aspects, the sulfur of the formulation is a colloidal sulfur. In aspects, the colloidal sulfur is present in an amount greater than about 5%, such as at least about 6.5%, at least about 7.5%, at least about 8.5%, at least about 9%, at least about 9.5%, or at least about 10% (e.g., about 4%-12%, such as about 4.5-11.5%, such as about 5-10%). In aspects, compositions provided herein comprise one or more of a functional compound or mixture of two or more functional compounds in an amount that is effective for detectably or significantly modifying one or more functional properties of the formulation. Examples of such functional compounds/mixtures include silicone-based polymer(s), such as polymeric organosilicon compound(s) (“silicone(s)”), such as polydimethylsiloxane, also known as dimethylpolysiloxane, i.e., dimethicone; a squalene or squalene-derivative, such as squalene, or functionally similar component in terms of its emollient properties (and in aspects reduction of itch, irritation, or both). In aspects, such functional compounds/mixtures are characterizable as, e.g., emollient(s), surfactant(s), astringent(s), oil-, e.g., sebum-absorbant(s), drying agent(s), texturizer(s), thickener(s)/viscosity enhancer(s), binding agent(s), conditioner(s), moisturizer(s), humectant(s), anti-pruritic (anti-itch) agent(s), stabilizing agent(s), emulsifier(s), lubricant(s), or barrier-forming or protection agent(s). In certain respects, any one or more such compounds or mixtures of compound(s) can comprise long chain fatty acids and can comprise any one or more such functions. In certain response, any one or more such compounds or mixtures of compound(s) can comprise any two or more such functional characteristics. In aspects, compositions are topically applied to skin in the form of a cream.

In aspects, the invention describes topical cream compositions comprising sulfur as a single active ingredient, present in the composition along with one or more of a compound or mixture of two or more compounds characterizable as emollient(s), surfactant(s), astringent(s), oil-, e.g., sebum-absorbent(s), drying agent(s), texturizer(s), thickener(s)/viscosity enhancer(s), binding agent(s), conditioner(s), moisturizer(s), humectant(s), anti-pruritic (anti-itch) agent(s), stabilizing agent(s), emulsifier(s), lubricant(s), or barrier-forming or protection agent(s) whereby the composition provides detectable or significant anti-fungal activity, anti-yeast activity, anti-bacterial activity, anti-pruritic activity, or anti-inflammatory activity. In some aspects, the compositions described herein are capable of providing two or more of, three or more of, or each of detectable or significant anti-fungal activity, anti-yeast activity, anti-bacterial activity, anti-pruritic activity, and anti-inflammatory activity.

In certain aspects, the invention describes compositions suitable for topical application to the skin of a mammal, e.g., a human, comprising sulfur as the sole active and further comprising long carbon chain compounds, such as long chain fatty acid compounds and one or more other functional compounds, whereby the composition(s) is suitable for the treatment of any one or more of acne, pimples, rosacea, seborrheic dermatitis (dandruff), perioral dermatitis, tinea versicolor, pityrosporon folliculitis, and mild dermatitis such as that which is caused by insect bite(s).

In certain facets, the compositions are topical creams such as those described above comprising the anti-pruritic Calamine or functionally equivalent ingredients to Calamine such that the invention describes compositions capable of treating any one or more of acne, pimples, rosacea, seborrheic dermatitis, perioral dermatitis, tinea versicolor, pityrosporon folliculitis, and mild dermatitis while concurrently being capable of treating mild itchiness from any of such conditions or from, e.g., sunburn, insect bite(s), poison ivy, or other mild skin conditions.

In certain facets, the compositions of the invention are capable of treating such conditions such as acne, pimples, rosacea, seborrheic dermatitis, perioral dermatitis, tinea versicolor, pityrosporon folliculitis, and mild dermatitis without causing detectable or significant scabbing of the underlying skin.

In certain respects, the invention provides topical compositions comprising sulfur as the sole active ingredient and further comprising long carbon chain compounds, such as long chain fatty acid compounds and one or more other functional compounds which are non-comedogenic. In some facets, the invention provides topical compositions comprising sulfur as the sole active agent and further comprising long carbon chain compounds, such as long chain fatty acid compounds and one or more other functional compounds comprising detectable or significant anti-fungal activity, anti-yeast activity, anti-bacterial activity, anti-pruritic activity, or anti-inflammatory activity while providing cosmetically desirable characteristics such as having in aspects mattifying properties, non-sticky feel or other pleasant skin feel, acceptable odor, pleasant appearance, or a combination thereof, as reported by a significant number of users, such as at least about 50% of users in an appropriately conducted survey or study.

In certain facets, the invention describes compositions comprising sulfur in the form of colloidal sulfur, provided in a cream formulation for topical application to the skin of a mammal, the colloidal sulfur present in the composition along with one or more of a compound or mixture of two or more compounds characterizable as emollient(s), surfactant(s), astringent(s), oil-, e.g., sebum-absorbant(s), drying agent(s), texturizer(s), thickener(s)/viscosity enhancer(s), binding agent(s), conditioner(s), moisturizer(s), humectant(s), anti-pruritic (anti-itch) agent(s), stabilizing agent(s), emulsifier(s), lubricant(s), or barrier-forming or protection agent(s), whereby the composition provides detectable or significant anti-fungal activity, anti-yeast activity, anti-bacterial activity, anti-pruritic activity, and anti-inflammatory activity, and further whereby the composition(s) is suitable for the treatment of any one or more of acne, pimples, rosacea, seborrheic dermatitis (dandruff), perioral dermatitis, tinea versicolor, pityrosporon folliculitis, and mild dermatitis.

According to some aspects, the invention provides methods of manufacturing any of the compositions described above.

According to aspects the invention provides methods of treating or preventing diseases or conditions of the skin, such as acne or rosacea, comprising delivering to the skin of a mammal, such as a human patient, an effective amount of one or more of the sulfur-containing compositions described herein, in a sufficient number of application(s) per treatment period, for a sufficient period of time, so as to detectably or significantly treat the disease or condition. In aspects, such methods are associated with detectably or measurably enhanced patient compliance, detectably or measurably enhanced improvement in one or more aspects of treatment (such as lack of or treatment of itching in the patient), detectably or measurably reduced adverse events in patients, or a combination of some or all thereof, when compared with the performance of other on-the-market or described topical sulfur skincare products.

### DETAILED DESCRIPTION OF THE INVENTION

Disclosed herein are compositions suitable for topical application to the skin of a mammal for the purpose of treating or preventing a skin condition, such as acne or also or alternatively, for daily use in the maintenance of healthy skin comprising a single active ingredient, present in the composition along with one or more long chain carbon compounds, such as long chain fatty acids or fatty acid compounds.

In aspects, compositions described herein when applied in effective amounts for a suitable period of time and number of applications per treatment period detectably or significantly treat or prevent acne (e.g., bacterial acne (acne vulgaris) or fungal acne), rosacea, acne rosacea, psoriasis (e.g., scap psoriasis), eczema, seborrheic dermatitis, scabies, rashes, hayfever, seborrhea, dandruff, and scars/scarring, or other conditions, symptoms, etc., known to be treated by topical sulfur (e.g., reducing inflammatory lesions & perilesional erythema of papulopustular rosacea or reduction of facial seborrheic dermatitis in patients with “rosacea-seborrheic dermatitis overlap”).

In aspects, the compositions provided herein comprise one or more of a functional compound or mixture of two or more functional compounds characterizable as emollient(s), surfactant(s), astringent(s), oil-, e.g., sebum-absorbant(s), drying agent(s), texturizer(s), thickener(s)/viscosity enhancer(s), binding agent(s), conditioner(s), moisturizer(s), humectant(s), anti-pruritic (anti-itch) agent(s), stabilizing agent(s), emulsifier(s), lubricant(s), or barrier-forming/protection agent(s).

In certain respects, any one or more such compounds or mixtures of compound(s) can comprise one or more long chain fatty acid compound(s) that provide any one or more such functions described above. In aspects, compositions comprise two or more long chain fatty acid compounds (fatty acids or fatty acid derivatives, such as fatty acid alcohols). In aspects, one, some, most, generally all, or all of such fatty acid compounds are C16 or C18 compounds. In aspects, one, some, most, generally all, or all such fatty acid compounds are C18 compounds, such as various stearate compounds, e.g., stearic acid, stearate containing compositions, such as glyceryl stearate, or stearate derivatives, such as PEGylated stearates, e.g., PEG-100 stearate.

In certain response, any one or more such compounds or mixtures of compound(s) can comprise any two or more such functional characteristics. In certain further aspects, the compositions of the invention provide detectable or significant anti-fungal activity, anti-yeast activity, anti-bacterial activity, anti-pruritic activity, or anti-inflammatory activity. In some respects, the compositions described herein are capable of providing some, most, or each of detectable or significant anti-fungal activity, anti-yeast activity, anti-bacterial activity, anti-pruritic activity, and anti-inflammatory activity, and are provided in formulations suitable for topical application to the skin.

In aspects, water makes up a substantial proportion of a formulation of the invention, such as at least about 20%, at least about 25%, at least about 33%, or at least about 35% of the formulation. Compositions described herein can comprise water, e.g., deionized water, in an amount of at least about 40%, such as at least about 42%, at least about 44%, at least about 46%, at least about 48%, or at least about 50%. According to certain aspects, compositions described herein are primarily water-based (i.e., aqueous) compositions. In exemplary aspects, formulations of the invention can comprise about 52%, at least about 54%, at least about 56%, at least about 58%, or, e.g., at least about 60%. In aspects, formulations comprise between about 40% to about 60% water, such as between about 42-about 60% water, between about 44-about 58% water, between about 46-about 56% water, or between about 48-about 56% water, such as between about 50-about 56% water, for example between about 52-about 55% water or, e.g., between about 53-about 55% water, such as about 54% water. In certain specific aspects, compositions of the invention comprise about 54.33% deionized water.

According to certain embodiments, compositions of the invention are suitable for the topical application to mammalian skin, such as to the skin of a human. In some aspects, such compositions can be epicutaneous. In certain facets, delivered in any suitable form such as, for example, solutions, gels, creams, lotions, pastes, ointments, aerosols, foams, sprays, powders, solids, or patches. In certain specific aspects, the compositions are not foams or aerosolized compositions. In certain aspects, the composition is in the form of a lotion or a cream. In aspects, the composition is classifiable as a cream.

Compositions of the invention comprise sulfur as an active ingredient. In aspects, compositions of the invention comprise sulfur as the single active ingredient. In certain respects, compositions of the invention are aqueous solutions comprising a sulfur. In facets, the sulfur is present as elemental sulfur, precipitated sulfur, colloidal sulfur, or a combination thereof. In common embodiments, the sulfur is present as a colloidal sulfur. In one aspect, colloidal sulfur is the primary colloid in the composition. In aspects, colloidal sulfur is the only colloid in the composition.

According to some embodiments, sulfur is present in the composition in an amount of at least about 5%, such as at least about 5.5%, at least about 6%, at least about 6.5%, at least about 7%, at least about 7.5%, at least about 8%, at least about 8.5%, at least about 9%, at least about 9.5%, at least about 10%, or even more, such as at least about 10.5%, at least about 11%, at least about 11.5%, at least about 12%, at least about 12.5%, at least about 13%, at least about 13.5%, at least about 14%, at least about 14.5%, at least about 15%, or even higher. In certain aspects, sulfur is present in the composing in an amount between about 5-about 15%, such as between about 6-about 14%, between about 7-about 13%, between about 8-about 12%, between about 9-about 11%, or for example sulfur is present in an amount of about 10%. In one aspect, sulfur is present as colloidal sulfur, the sulfur itself present in an amount of about 10.01% of the composition.

In certain aspects, sulfur can be provided in colloid form. In certain respects, the second compound forming the colloid with sulfur as the first compound can be any dermatologically/cosmetically safe compound which does not prevent the composition from being suitable for topical application. In aspects, such colloid compounds can be, for example, albumin, dextran, hydroxyethyl starches (HES), including tetrastarches. In aspects, the colloid is a hydrophilic polymer (e.g., hydrocolloid). In aspects the colloid compounds can be natural polysaccharides, e.g., agar, carrageenan, gelatin, or pectin. In aspects, the colloid compound can be a gum, such as xanthan gum, gum arabic, guar gum, locus bean gum, or for example a cellulose derivative such as carboxymethyl cellulose, alginate, or starch. In certain specific aspects, the sulfur is present as a colloidal sulfur in Acacia Senegal Gum.

In some aspects, the colloid compound can be present in an amount of between about 1% and about 10% of the composition, such as between about 1% and about 9%, between about 1% and about 8%, between about 1% and about 7%, between about 1% and about 6%, between about 1 and about 5%, or between about 2% and about 4%. In certain aspects, the colloid compound can comprise about 3% of the composition. In one facet, the composition comprises Acacia Senegal Gum in an amount of about 3%, such as in one particular embodiment in an amount of about 3.34%.

According to some aspects, colloidal sulfur can be provided as a single ingredient. In some aspects, the composition comprises colloidal sulfur provided as a single ingredient, the single ingredient being present in an about of between about 5% and about 25%, such as between about 7-about 23%, between about 9-about 21%, between about 11-about 19%, or between about 12%-about 18%, such as between about 12%-about 16%, or between about 12%-about 14%. In some aspects, the colloidal sulfur ingredient is present in the composition in an amount of about 13%. In one aspect, the colloidal sulfur is provided as Sulfidal® (sulfur and Acacia Senegal gum) in an amount of about 13.35%, wherein 75% of Sulfidal® is sulfur, such that Sulfidal® in an amount of 13.35% provides sulfur in an amount that represents about 10.01% of the total composition. According to some facets, compositions described herein comprise at least 12% of a colloidal sulfur ingredient that comprises at least about 60% sulfur, such as at least about 62% sulfur, at least about 64% sulfur, at least about 66% sulfur, at least about 68% sulfur, at least about 70% sulfur, at least about 72% sulfur, at least about 74% sulfur, at least about 76% sulfur, at least about 78% sulfur, or, e.g., at least about 80% sulfur. In some aspects, compositions described herein comprise at least 12% of a colloidal sulfur ingredient wherein use of the ingredient provides a composition comprising approximately 10% sulfur.

According to certain embodiments, sulfur provides one or more of drying or sebum-absorbing properties. In certain aspects, sulfur also or alternatively provides one or more of detectable or significant anti-fungal, anti-bacterial, or keratolytic properties to the compositions described herein.

In some aspects, sulfur, e.g., colloidal sulfur, is present along with one or more additional active ingredients. Such one or more additional active ingredients can provide one or more additional functions, including but not limited to detectable or significant anti-fungal activity, anti-yeast activity, anti-bacterial activity, anti-pruritic activity, or anti-inflammatory activity. In certain aspects, compositions described herein can comprise sulfur, e.g., colloidal sulfur, along with one or more additional active ingredients, and can further comprise a functional ingredient or excipient.

In some respects, sulfur, e.g., colloidal sulfur, is the single active ingredient and can be present in the composition along with one or more of a functional ingredient or excipient, e.g., a compound or mixture of two or more compounds. In aspects, such compounds can be characterizable as one or more of an emollient(s), surfactant(s), astringent(s), oil-, e.g., sebum-absorbent(s), drying agent(s), texturizer(s), thickener(s)/viscosity enhancer(s), binding agent(s), conditioner(s), moisturizer(s), humectant(s), anti-pruritic (anti-itch) agent(s), stabilizing agent(s), emulsifier(s), lubricant(s), and/or barrier-forming or protection agent(s).

In some aspects, compositions of the invention comprise colloidal sulfur and one or more functional elements which can be characterized as comprising long-chain hydrocarbons, in aspects characterizable as long-chain lipids, such as, in aspects, long chain fatty acids. In aspects, the compositions herein are rich in, e.g., comprise high amounts of, compounds having at least 16-carbon chains. For example, the compositions described herein can comprise compounds (or ingredients comprising mixtures of compounds) comprising carbon chains of at least 16 carbons, such compounds or mixtures of compounds representing at least about 16% of the composition, such as at least about 17%, at least about 18%, at least about 19%, or at least about 20% of the composition, such as at least about 21% or more of the composition. In certain aspects, such compounds or mixtures of compounds represent about 20.5% of the composition. In certain aspects, compositions of the present invention comprise ingredients such as squalane, dicapyrlyl carbonate, sunflower seed oil, PEG-100 stearate, shea butter glyceride mixtures, stearic acid, cetearyl alcohol, and an ingredient comprising a mixture of pentylene glycol, butylene glycol, and hydroxyphenyl propamidobenzoic acid, these ingredients contributing compounds or mixtures of compounds comprising 16-carbons or more and making up at least about 15%, such as at least about 16%, at least about 17%, at least about 18%, at least about 19%, or for example at least approximately 20% of the compositions described herein. In certain aspects, such compounds or mixtures of compounds represent approximately 20.5% of the compositions described herein.

As described, compositions of the present invention can comprise compounds, or mixtures of compounds (e.g., single ingredients comprising a mixtures of compounds) which comprise long chain hydrocarbons, e.g., comprising at least 16 carbons. In some aspects, such long hydrocarbon chain-comprising ingredient can be any single or mixture of ingredients comprising hydrocarbon chain(s) having at least 16 carbons.

In one aspect, compositions described herein comprise at least about 1% squalane, such as at least about 1.2%, at least about 1.4%, at least about 1.6%, at least about 1.8%, or at least about 2% squalane, such as for example at least approximately 2.2% squalane, at least approximately 2.4% squalane, at least approximately 2.6% squalane, at least approximately 2.8% squalane, at least approximately 3% squalane or more, such as at least about 3.2%, at least about 3.4%, at least about 3.6%, at least about 3.8%, or at least about 4% squalane. According to some embodiments, compositions of the invention comprise between approximately 1%-approximately 6% squalane, such as, e.g., between approximately 1.5%-approximately 5.5% squalane, between approximately 2%-approximately 5% squalane, between approximately 2.5%-approximately 4.5% squalane, between approximately 2.5%-approximately 4% squalane, or between approximately 2.5%-approximately 3.5% squalane. In certain facets, compositions described herein comprise approximately 3% squalane. In certain facets, squalane can be provided in the form of Neossance™ Squalane in an amount of about 3%. In some aspects, squalane provides emollient properties which in certain aspects provides any one or more of detectable or significant softening, moisturizing, and anti-itch properties.

In one aspect, compositions described herein comprise cetearyl alcohol. In one facet, compositions described herein comprise ceteareth-20. In certain embodiments, compositions described herein comprise both cetearyl alcohol and ceteareth-20, each present in an amount of less than about 3% of the composition, such as compositions comprising less than about 2.9%, less than about 2.8%, less than about 2.7%, less than about 2.6%, less than about 2.5%, less than about 2.4%, less than about 2.3%, less than about 2.2%, or less than about 2.1%, such as less than about 2% of either cetearyl alcohol or ceteareth-20. In aspects, the compositions comprise cetearyl alcohol and ceteareth-20 wherein the two compounds are provided as a single ingredient representing between approximately 0.5% to approximately 3.5% of the composition, such as between approximately 0.6%-approximately 3.4%, between approximately 0.8%-approximately 3.2%, between approximately 1%-approximately 3%, between approximately 1.2%-approximately 2.8%, between approximately 1.4%-approximately 2.6%, between approximately 1.6%-approximately 2.4%, or for example between approximately 1.8%-approximately 2.2%. In certain aspects, compositions of the invention comprise cetearyl alcohol and ceteareth-20 in an amount representing between about 1.8%-about 2.2%, such as approximately 2% of the composition, whether added individually or as a single ingredient. In certain aspects, the compounds are provided as a single ingredient, the single ingredient being Promulgen™ D and present in an amount representing approximately 2% of the composition. In some aspects, any one of the compounds alone, or the compounds in combination, whether added individually or as a single ingredient, provide detectable or significant emulsification properties.

In some aspects, compositions described herein comprise pentylene glycol. In some aspects, compositions described herein comprise butylene glycol. In some aspects, compositions described herein comprise hydroxyphenyl propamidobenzoic acid. In some aspects, compositions described herein comprise pentylene glycol, butylene glycol, and propamidobenzoic acid. In certain facets, each of pentylene glycol, butylene glycol, and propamidobenzoic acid are present in an amount representing less than about 3% of the composition, such as less than about 2.8%, less than about 2.6%, less than about 2.4%, less than about 2.2%, or for example less than about 2% of the composition. In some aspects, the three compounds (pentylene glycol, butylene glycol, and propamidobenzoic acid) are provided as a single ingredient representing between about 0.5% to about 3.5% of the composition, such as between about 0.6%-about 3.4%, between about 0.8%-about 3.2%, between about 1%-about 3%, between about 1.2%-about 2.8%, between about 1.4%-about 2.6%, between about 1.6%-about 2.4%, or for example between about 1.8%-about 2.2%. In certain aspects, compositions of the invention comprise pentylene glycol, butylene glycol, and propamidobenzoic acid, the three compounds together, whether added individually or as a single ingredient, present in an amount of between approximately 1.8%-2.2% of the compositions, such as about 2% of the composition. In aspects, the compositions described herein comprise the three compounds provided as a single ingredient and wherein the single ingredient is present in an amount representing between about 1.8-approximately 2.2%, such as representing approximately 2% of the composition. In certain aspects, the single ingredient is SymCalmin® 143535, present in an amount representing approximately 2% of the composition. In some aspects, any of the compounds, or any combination thereof, whether added individually or together as a single ingredient, provide any one or more of detectable or significant anti-irritant, anti-itch, anti-histaminic, anti-inflammatory, anti-redness, or anti-oxidant activity.

In certain aspects, compositions described herein comprise dicaprylyl carbonate. In one aspect, compositions described herein comprise at least about 1% dicaprylyl carbonate, such as at least about 1.2%, at least about 1.4%, at least about 1.6%, at least about 1.8%, or at least about 2% dicaprylyl carbonate, such as for example at least approximately 2.2% dicaprylyl carbonate, at least approximately 2.4% dicaprylyl carbonate, at least approximately 2.6% dicaprylyl carbonate, at least approximately 2.8% dicaprylyl carbonate, at least approximately 3% dicaprylyl carbonate or more, such as at least about 3.2%, at least about 3.4%, at least about 3.6%, at least about 3.8%, or at least about 4% dicaprylyl carbonate, or, e.g., at least approximately 4.2%, at least approximately 4.4%, at least approximately 4.6%, at least approximately 4.8%, or at least approximately 5% dicaprylyl carbonate. In certain facets, compositions described herein comprise between about 2%-about 6% dicaprylyl carbonate, such as between about 2.5%-about 5.5%, between about 3%-about 5%, or between about 3.5%-about 4.5% dicaprylyl carbonate. In certain facets, compositions described herein comprise approximately 4% dicaprylyl carbonate. In certain facets, dicaprylyl carbonate can be provided in the form of Cetiol® in an amount of about 4%. In some aspects, dicaprylyl carbonate provides detectable or significant conditioning or emollient properties, such as e.g. in aspects providing detectable or significant softening or moisturizing properties.

In some aspects, the long chain hydrocarbons of the present invention can comprise long chain fatty acids. In some aspects, such long chain fatty acids can comprise at least 17 carbons or 18 carbons. In certain aspects, compositions of the present invention can be characterized as rich in long chain fatty acids, such as comprising compounds or combinations of compounds wherein the compounds or combinations of compounds comprise fatty acids having at least 17 carbons, such as 17 or 18 carbons. In certain aspects, long chain fatty acids can be present in an amount of at least about 5%, such as at least about 5.5%, at least about 6%, at least about 6.5%, at least about 7%, at least about 7.5%, at least about 8%, at least about 8.5%, at least about 9%, at least about 9.5%, at least about 10%, at least about 10.5%, at least about 11%, at least about 11.5%, at least about 12%, at least about 12.5%, at least about 13%, at least about 13.5%, or for example, at least approximately 14%, at least approximately 14.5%, or at least approximately 15%. In aspects, long chain fatty acids can be present in an amount representing between about 2%-about 20%, such as between about 4%-about 18%, between about 4%-about 16%, between about 6%-about 14%, or, e.g., between about 8%-about 12%, such as approximately 8.5%, approximately 9%, approximately 9.5%, approximately 10%, approximately 10.5%, approximately 11%, approximately 11.5%, or approximately 12% of the compositions described herein. In some aspects, such long chain fatty acids can be provided by Helianthus Annus (Sunflower) oil, polyethylene glycol (PEG)-100 stearate, shea butter glycerides, and, e.g., stearic acid, or combinations thereof.

In some aspects, compositions of the invention can be characterized as being rich in stearate compounds. In certain embodiments, compositions described herein can comprise compounds or mixtures of compounds which comprise stearate compounds and which represent at least about 4%, at least about 4.2%, at least about 4.4%, at least about 4.6%, at least about 4.8%, at least about 5%, at least about 5.2%, at least about 5.4%, at least about 5.6%, at least about 5.8%, at least about 6%, at least about 6.2%, at least about 6.4%, at least about 6.6%, at least about 6.8%, or, e.g., at least about 7% of the composition, or even more. In certain respects, the compositions of the invention can be characterized as comprising between about 1%-about 10% of stearate compounds, such as between about 2%-about 9% of stearate compounds, between about 3%-about 8% of stearate compounds, between about 4%-about 7% of stearate compounds, such as between about 5%-about 7% stearate compounds. Exemplary stearate compounds can comprise or be contributed by, e.g., polyethylene glycol (PEG)-100 stearate, shea butter glycerides, and, e.g., stearic acid.

According to some aspects, compositions described herein comprise Helianthus Annus (Sunflower) seed oil in an amount of at least about 1%, such as at least about 1.5%, at least about 2%, at least about 2.5%, at least about 3%, at least about 3.5%, at least about 4%, at least about 4.5%, at least about 5%, at least about 5.5%, at least about 6%, at least about 6.5%, or at least about 7%. In some aspects, compositions described herein comprise sunflower seed oil in an amount of between approximately 1-approximately 10%, such as between approximately 2%-approximately 9%, between approximately 3%-approximately 8%, between approximately 4%-approximately 7%, between approximately 4%-approximately 6%, such as approximately 5% sunflower seed oil. In certain aspects, the sunflower oil is a high oleic acid sunflower oil, e.g., having a minimum of 80% oleic acid. In some aspects, sunflower oil comprising at least 80% oleic acid is present in the composition in an amount representing approximately 5% of the composition. In some aspects, sunflower oil provides detectable or significant emollient properties, such as e.g. in aspects providing detectable or significant softening or moisturizing properties.

In certain facets, compositions described herein comprise polyethylene glycol (PEG)-100 stearate. In some aspects, compositions described herein comprise glyceryl stearate. In certain embodiments, compositions of the invention comprise both PEG-100 stearate and glyceryl stearate, each in an amount of less than about 3%, such as less than about 2.8%, less than about 2.6%, less than about 2.4%, or, e.g., less than about 2.2%, less than about 2%, less than about 1.8%, or e.g., less than about 1.6%. In some aspects, compositions of the invention comprise both PEG-100 stearate and glyceryl stearate, wherein the two ingredients are provided as a single ingredient. According to some facets. PEG-100 stearate and glyceryl stearate can be provided as a single ingredient wherein the single ingredient represents between approximately 1%-approximately S% of the compositions of the invention, e.g., between approximately 1.5%-approximately 4.5%, or between approximately 2%-approximately 4%, such as between approximately 2.2%-approximately 3.5%, between approximately 2.4%-approximately 3%, between approximately 2.4%-approximately 2.8%, or, for example, between approximately 2,4%-approximately 2.6%. In some aspects, whether provided individually or as a single ingredient, the compositions described herein comprise the two compounds in an amount of between about 2.4% and about 2.6%, such as approximately 2.5%. In some aspects, PEG-100-stearate and glyceryl stearate are provided as a single ingredient, the single ingredient present in an amount of approximately 2.4%-approximately 2.6%. In certain specific aspects, PEG-100 stearate and glyceryl stearate are provided as a single ingredient, the single ingredient being Simulsol™ 165, Simulsol™ 165 being present in an amount representing about 2.5% of the compositions. According to certain aspects, PEG-100 stearate, glyceryl stearate, or the combination of the two compounds, whether added individually or as a single ingredient, can provide surfactant or emulsification properties, can detectibly or significantly enhance product aesthetics (e.g., detectably or significantly enhance skin feel, evaluation of cosmetic elegance, and the like), as measured by customer survey or appropriately conducted study.

In some aspects, compositions described herein can comprise shea butter glycerides. In certain respects, shea butter glycerides can represent at least approximately 1%, e.g., at least about 1.2%, at least about 1.4%, at least about 1.6%, at least about 1.8%, at least about 2%, at least about 2.2%, at least about 2.4%, at least about 2.6%, at least about 2.8%, at least about 3%, at least about 3.2%, at least about 3.4%, at least about 3.6%, at least about 3.8%, or e.g., at least about 4% of the composition. In certain aspects, compositions described herein can comprise between about 1%-about 4% shea butter glycerides, such as between about 1.2%-about 3.6% shea butter glycerides, between about 1.4%-about 3.2% shea butter glycerides, between about 1.6%-about 2.8% shea butter glycerides, between about 1.8%-about 2.4% shea butter glycerides, or, e.g., between about 1.8%-about 2.2% shea butter glycerides. In aspects, compositions of the invention can comprise approximately 2% shea butter glycerides. In a specific embodiment, compositions described herein can comprise shea butter glycerides provided as Jarplex™ SB-WD, in an amount representing 2% of the composition. In some respects, shea butter glycerides can provide detectable or significant emollient, emulsification, or surfactant properties to the compositions.

In one aspect, compositions described herein comprise stearic acid. In certain aspects, compositions of the invention comprise at least about 1% stearic acid, such as at least about 1.2%, at least about 1.4%, at least about 1.6%, at least about 1.8%, or at least about 2% stearic acid, such as for example at least approximately 2.2% stearic acid, at least approximately 2.4% stearic acid, at least approximately 2.6% stearic acid, at least approximately 2.8% stearic acid, or at least approximately 3% stearic acid or more. In certain facets, compositions described herein comprise between about 1%-about 3% stearic acid, such as between about 1.2%-about 2.8%, between about 1.4%-about 2.6%, between about 1.6%-about 2.4%, or, e.g., between about 1.8%-about 2.2% stearic acid. In certain facets, compositions of the present invention comprise about 2% stearic acid. In some respects, a stearic acid ingredient or related long chain fatty acid compound ingredient provides detectable or significant cleansing properties or protective properties, e.g., detectably or significantly enhances the composition(s) ability to maintain skin moisture and prevent water loss.

In some aspects, compositions of the invention comprise hydroxyethyl acrylate. In certain facets, compositions of the invention comprise sodium acryloyldimethyl taurate copolymer. In some aspects, compositions of the invention comprise both hydroxyethyl acrylate and acryloyldimethyl taurate copolymer, each in an amount representing less than about 2% of the composition, such as less than about 1.8% of the composition, less than about 1.6% of the composition, less than about 1.4% of the composition, less than about 1.2% of the composition, or less than about 1% of the composition. In certain aspects, hydroxyethyl acrylate and acryloyldimethyl taurate copolymer can be provided as a single ingredient, the single ingredient provided in an amount representing at least about 0.5%, at least about 0.6%, at least about 0.7%, at least about 0.8%, at least about 0.9%, at least about 1%, at least about 1.1%, at least about 1.1%, at least about 1.2%, about 1.3%, at least about 1.4%, or at least about 1.5% of the composition. In aspects, hydroxyethyl acrylate and acryloyldimethyl taurate are provided as a single ingredient representing between approximately 0.5%-approximately 1.5%, between approximately 0.6%-approximately 1.4%, between approximately 0.7%-approximately 1.3%, between approximately 0.8%-approximately 1.2%, or, e.g., between approximately 0.9%-approximately 1.1%. In some facets, compositions can comprise hydroxyethyl acrylate and acryloyldimethyl taurate copolymer in an amount of between about 0.9%-1.1%, whether each compound is added individually or provided as a single ingredient. In certain aspects, compositions can comprise hydroxyethyl acrylate and acryloyldimethyl taurate copolymer provided as a single ingredient representing about 1% of the composition. In one specific aspect, the single ingredient is Sepinov™ EMT 10, present in an amount representing about 1% of the composition. In certain aspects, hydroxyethyl acrylate and acryloyldimethyl taurate copolymer, whether added individually or provided as a single ingredient, provide detectable or significant stabilizing_{;} thickening (e.g., viscosity-enhancing), or texturizing characteristics to the composition(s).

In one aspect, compositions described herein comprise glycerin. In certain aspects, compositions described herein comprise at least about 1% glycerin, such as at least about 1.2%, at least about 1.4%, at least about 1.6%, at least about 1.8%, or at least about 2% glycerin, such as for example at least approximately 2.2% glycerin, at least approximately 2.4% glycerin, at least approximately 2.6% glycerin, at least approximately 2.8% glycerin, or at least approximately 3% glycerin or more, such as for example at least approximately 3.2%, at least approximately 3.4%, at least approximately 3.6%, at least approximate 3.8%, or at least about 4%, such as at least about 4.2%, at least about 4.4%, at least about 4.6%, at least about 4.8%, or at least about 5% glycerin. In certain facets, compositions described herein comprise between about 1%-about 5% glycerin, such as between about 1.2%-about 4.8%, between about 1.4%-about 4.6%, between about 1.6%-about 4.4%, or, e.g., between about 1.8%-about 4.2% glycerin, e.g., between about 2%-about 4% glycerin, between about 2.2%-about 3.8% glycerin, between about 2.4%-about 3.6% glycerin, between about 2.6%-about 3.4% glycerin, or between about 2.8%-about 3.2% glycerin. In certain facets, compositions of the present invention comprise about 3% glycerin. In one aspect, the glycerin is provided as 99.7% USP grade glycerin. In some respects, glycerin provides detectable or significant humectant or moisturizing properties to the composition.

In one embodiment, compositions of the invention comprise at least one silicone-based ingredient. In certain aspects, the one or more silicone-based ingredient(s) can detectably or significantly improve the composition(s) ability to influence the hydration of the skin of the composition recipient. In certain aspects, one or more silicones can also or alternatively detectably or significantly improve one or more aesthetic properties of the composition, such as for example but not limited to providing matting properties (e.g., reduction in shine). In certain aspects, the presence of one or more silicones in the composition can detectably or significantly blur or reduce the appearance of skin pores or also or alternatively detectably or significantly reduce the appearance of lines and wrinkles, e.g., for a period of at least about 30 minutes, about 1 hour, about 1.5 hours, about 2 hours, about 2.5 hours, about 3 hours, about 4 hours, about 5 hours, about 6 hours, about 8 hours, about 10 hours, about 12 hours, about 15 hours, about 20 hours, or e.g., for about 24 hours, or longer.

In certain aspects, the one or more silicones can be any silicone compound suitable for safe and effective application to mammalian skin, such as but not limited to dimethicone, cyclomethicone, cyclohexasiloxane, ceteryl methiocone, or cyclopentasiloxane.

In one aspect, compositions described herein comprise dimethicone. In certain aspects, compositions described herein comprise at least about 1% dimethicone, such as at least about 1.2%, at least about 1.4%, at least about 1.6%, at least about 1.8%, or at least about 2% dimethicone, such as for example at least approximately 2.2% dimethicone, at least approximately 2.4% dimethicone, at least approximately 2.6% dimethicone, at least approximately 2.8% dimethicone, or at least approximately 3% dimethicone or more. In certain facets, compositions described herein comprise between about 1%-about 4% dimethicone, such as between about 1.2%-about 4.6%, between about 1.4%-about 4.2%, between about 1.6%-about 3.8%, or, e.g., between about 1.8%-about 3.4% dimethicone, e.g., between about 1.8%-about 3% dimethicone, between about 1.8%-about 2.6% dimethicone, or between about 1.8%-about 2.2% dimethicone. In certain facets, compositions of the present invention comprise about 2% dimethicone. In one aspect, the dimethicone is provided as Dimethisil® DM-350. In some aspects, dimethicone provides detectable or significant protectant, moisturizing, or emollient properties to the composition, such as, e.g., detectably or significantly enhancing the composition(s) ability to maintain moisture and prevent water loss, or e.g., provides any one or more characteristics described above for silicone-based ingredients.

In one aspect, compositions described herein comprise an ingredient provided to detectably or significantly reduce pruritis or itching. In certain aspects, compositions of the invention comprise calamine. In certain aspects, compositions of the invention comprise the active ingredients present in calamine in amounts representative of a calamine formulation, such as, e.g., zinc oxide and ferric oxide. In certain aspects, compositions described herein comprise zinc oxide and ferric oxide, each in an amount of less than about 3% of the composition, such as each in an amount of less than about 2.8%, less than about 2.6%, less than about 2.4%, less than about 2.2%, or less than about 2%, such as less than about 1.8% or less than about 1.6%. In certain aspects, zinc oxide and ferric oxide are present and provided as a single ingredient, such as the single ingredient calamine. In certain aspects, compositions described herein comprise at least about 1% calamine, such as at least about 1.2%, at least about 1.4%, at least about 1.6%, at least about 1.8%, or at least about 2% calamine, such as for example at least approximately 2.2% calamine, at least approximately 2.4% calamine, at least approximately 2.6% calamine, at least approximately 2.8% calamine, or at least approximately 3% calamine or more. In certain facets, compositions described herein comprise between about 1%-about 4% calamine, such as between about 1.2%-about 4.6%, between about 1.4%-about 4.2%, between about 1.6%-about 3.8%, or, e.g., between about 1.8%-about 3.4% calamine, e.g., between about 1.8%-about 3% calamine, between about 1.8%-about 2.6% calamine, or between about 1.8%-about 2.2% calamine. In certain facets, compositions of the present invention comprise about 2% calamine, or the compounds zinc oxide and ferric oxide are provided as individual ingredients, the two ingredients together representing approximately 1.5%-approximate 2%, such as approximately 1.4%-approximately 2%, approximately 1.6%-approximately 2%, or, e.g., approximately 1.8%-approximately 2% of the composition. In one aspect, calamine is provided as calamine powder. In some aspects, zinc oxide provides detectable or significant astringent properties to the composition. In some aspects, ferric oxide provides antipruritic properties to the composition. In some aspects, the combination of ferric oxide and zinc oxide, whether added individually or as the single ingredient calamine, provide detectable or significant anti-itch/anti-pruritic properties to the composition(s).

In one aspect, compositions described herein comprise vitamin E acetate. In certain aspects, compositions described herein comprise at least about 0.1% vitamin E acetate, such as at least about 0.15%, at least about 0.2%, at least about 0.25%, at least about 0.3%, or at least about 0.35% vitamin E acetate, such as for example at least approximately 0.4% vitamin E acetate, at least approximately 0.45% vitamin E acetate, or at least approximately 0.5% vitamin E acetate or more. In certain facets, compositions described herein comprise between about 0.1%-about 0.5% vitamin E acetate, such as between about 0.15%-about 0.45%, between about 0.2%-about 0.4%, between about 0.2%-about 0.4%, or, e.g., between about 0.25%-about 0.35% vitamin E acetate. In certain facets, compositions of the present invention comprise about 0.3% vitamin E acetate. In one aspect, the vitamin E acetate is provided as tocopheryl acetate. In some aspects, vitamin E acetate/tocopheryl acetate provides detectable or significant protectant, moisturizing, or emollient properties to the composition, such as, e.g., detectably or significantly enhancing the antioxidant property/ies of the composition(s).

In certain aspects, compositions of the invention comprise Magnolia Officinalis bark extract. In certain aspects, compositions of the invention comprise the active ingredients present in Magnolia Officinalis bark extract in amounts representative of a Magnolia Officinalis super extract (e.g., Magnolia Super Extract offered by Premier Specialties, Inc.), such as, e.g., one or more phenylpropanoids, such as, e.g., the biphenylpropanoid Magnolol or the biphenylpropanoid Honokiol, and also or alternatively one or more lignans, such as Magnone A and Magnone B-lignan. In certain aspects, compositions described herein comprise biphenylpropanoid(s) and lignan(s), each in an amount of less than about 0.3% of the composition, such as each in an amount of less than about 0.25%, less than about 0.2%, less than about 0.15%, or less than about 0.1%, such as less than about 0.08%, less than about 0.06% or less than about 0.04%. In certain aspects, Magnolol, Honokiol, Magnone A and Magnone B are present in the formulation and provided as a single ingredient, such as the single ingredient Magnolia Officinalis bark extract. In certain aspects, compositions described herein comprise at least about 0.05% Magnolia Officinalis bark extract, such as at least about 0.06%, at least about 0.07%, at least about 0.08%, at least about 0.09%, or at least about 0.1% Magnolia Officinalis bark extract, such as for example at least approximately 0.11% Magnolia Officinalis bark extract, at least approximately 0.12% Magnolia Officinalis bark extract, at least approximately 0.13% Magnolia Officinalis bark extract, at least approximately 0.14% Magnolia Officinalis bark extract, or at least approximately 0.15% Magnolia Officinalis bark extract or more. In certain facets, compositions described herein comprise between about 0.05%-about 0.15% Magnolia Officinalis bark extract, such as between about 0.06%-about 0.14%, between about 0.07%-about 0.13%, between about 0.08%-about 0.12%, or, e.g., between about 0.09%-about 0.11% Magnolia Officinalis bark extract. In certain facets, compositions of the present invention comprise about 0.1% Magnolia Officinalis bark extract, or the compounds Magnolol, Honokiol, Magnone A and Magnone are provided as individual ingredients, the four ingredients together representing approximately 0.05%-about 0.15%, such as between about 0.06%-about 0.14%, between about 0.07%-about 0.13%, between about 0.08%-about 0.12%, or, e.g., between about 0.09%-about 0.11% of the composition(s). In one aspect, Magnolia Officinalis bark extract is provided as Premier Magnolia Super Extract (Premier Specialties, Inc.). In some aspects, Magnolia Officinalis bark extract, or the components present therein previously discussed added individually, provides detectable or significant anti-oxidant (e.g., detectable or significant free radical scavenging) properties to the composition, anti-inflammatory properties to the composition(s), or both.

According to one facet, compositions of the invention can comprise one or more ingredients which act as a preservative. In certain aspects, such a one or more ingredient can, alone or in combination, provide detectible or significant anti-microbial activity, such as detectable or significant prevention of bacterial, fungal, yeast, or mold activity or detectable or significant reduction of microbial activity. According to certain embodiments, compositions of the invention can comprise phenoxyethanol. In certain aspects, compositions of the invention comprise caprylyl glycol. In some facets, compositions of the invention comprise hexylene glycol. In some embodiments, compositions of the invention comprise ethylhexylglycerin. In certain facets, compositions of the invention can comprise phenoxyethanol, caprylyl glycol, hexylene glycol, and ethylhexylglycerin, each present in an amount representing less than about 2% of the composition, such as less than about 1.9%, less than about 1.8%, less than about 1.7%, less than about 1.6%, less than about 1.5%, less than about 1.4%, less than about 1.3%, less than about 1.2%, less than about 1.1%, or less than about 1%, such as less than about 1.9%, or less than about 1.8%, or even less of the composition. In certain aspects, compositions of the invention can comprise phenoxyethanol, caprylyl glycol, hexylene glycol, and ethylhexylglycerin provided as a single ingredient, the single ingredient present in an amount of less than approximately 2% of the composition, such as less than approximately 1.8% of the composition, less than approximately 1.6% of the composition, less than approximately 1.4% of the composition, less than approximately 1.2% of the composition, or less than approximately 1% of the composition. In some aspects, compositions of the invention comprise phenoxyethanol, caprylyl glycol, hexylene glycol, and ethylhexylglycerin, wherein whether added individually or provided as a single ingredient, the four compounds comprise between about 0.2%-about 2%, such as between about 0.4%-about 1.8%, between about 0.6%-about 1.6%, between about 0.8%-about 1.4%, or between about 0.8%-about 1.2% of the composition. In certain aspects, phenoxyethanol, caprylyl glycol, hexylene glycol, and ethylhexylglycerin are provided as a single ingredient and comprise about 1% of the composition. In certain specific aspects, the single ingredient is Botanistat PF-64, present in an amount representing about 1% of the composition. In aspects, phenoxyethanol, caprylyl glycol, hexylene glycol, and ethylhexylglycerin, either alone or in combination, provide preservation characteristics, e.g., anti-microbial activity, such as detectable or significant prevention of bacterial, fungal, yeast, or mold activity or detectable or significant reduction of microbial activity.

According to certain embodiments, compositions of the invention can comprise Melaleuca alternifolia (tea tree) oil. In certain facets, compositions of the invention can comprise Melaleuca alternifolia (tea tree) oil in an amount representing less than about 0.1% of the composition, such as less than about 0.09⁰/0, less than about 0.08%, less than about 0.07%, less than about 0.06%, less than about 0.05%, less than about 0.04%, or, e.g., less than about 0.03% or even less of the composition. In certain aspects, compositions of the invention can comprise Melaleuca alternifolia (tea tree) oil present in an amount between about 0.01%-about 0.08%, such as between about 0.02%-about 0.07%, between about 0.03%-about 0.06%, or, for example, between about 0.03%-about 0.05% of the composition. In certain aspects, Melaleuca alternifolia (tea tree) oil comprises about 0.04% of the compositions described herein. In certain facets, Melaleuca alternifolia (tea tree) oil can provide preservation characteristics to the composition(s), e.g., anti-microbial activity, such as detectable or significant prevention of bacterial, fungal, yeast, or mold activity or detectable or significant reduction of microbial activity.

In one aspect, compositions described herein comprise bisabolol. In certain aspects, compositions described herein comprise at least about 0.1% bisabolol, such as at least about 0.15%, at least about 0.2%, at least about 0.25%, at least about 0.3%, or at least about 0.35% bisabolol, such as for example at least approximately 0.4% bisabolol or more. In certain facets, compositions described herein comprise between about 0.1%-about 0.3% bisabolol, or between about 0.15%-about 0.25% bisobolol. In certain facets, compositions of the present invention comprise about 0.2% bisabolol. In one aspect, the bisabolol provides detectable or significant fragrance or detectable or significant skin soothing, anti-microbial, antioxidant, or skin brightening properties to the composition(s).

In one aspect, compositions described herein can comprise one or more compounds having preservative or stabilizing properties. In one aspect, compositions described herein comprise disodium ethylenediaminetetraacetic acid (EDTA). In certain aspects, compositions described herein comprise at least about 0.01% disodium EDTA, such as at least about 0.02%, at least about 0.03%, at least about 0.04%, at least about 0.05%, or at least about 0.06%, at least about 0.07%, at least about 0.08%, at least about 0.09%, or e.g., at least about 0.1% disodium EDTA or more. In certain facets, compositions described herein comprise between about 0.01%-about 0.1% disodium EDTA, such as between about 0.02%-about 0.09%, between about 0.03%-about 0.08%, between about 0.04%-about 0.07%, or, e.g., between about 0.04%-about 0.06% disodium EDTA. In certain facets, compositions of the present invention comprise about 0.05% disodium EDTA. In one aspect, the disodium EDTA is provided as Dissolvine® NA2-P. In some respects, disodium EDTA provides detectable or significant chelating or stabilizing properties to the composition, such as, e.g., detectably or significantly enhancing the stability of the composition (e.g., preventing detectable or significant changes in composition pH, consistency, texture, smell/odor, appearance, or the like).

In one aspect, compositions described herein can comprise one or more ingredients serving solely as a fragrance-enhancing component. In certain aspects, compositions described herein comprise a fragrance (parfum). In certain aspects, compositions described herein comprise at least about 0.1% of a fragrance/parfum, such as at least about 0.11%, at least about 0.115%, at least about 0.12%, at least about 0.125%, or at least about 0.13% of a fragrance/parfum, such as for example at least approximately 0.135% fragrance/parfum, at least approximately 0.14%, or at least approximately 0.145% or at least approximately 0.15% fragrance/parfum. In certain facets, compositions described herein comprise between about 0.1%-about 0.2% fragrance/parfum, or between about 0.11%-about 0.18% fragrance/parfum, between about 0.12%-about 0.16% fragrance/parfum, or between about 0.12%-about 0.14% fragrance/parfum. In certain facets, compositions of the present invention comprise about 0.13% fragrance/parfum. In one specific aspect, the fragrance/parfum is “Apple of My Eye” UAC08799/00. In one aspect, the fragrance/parfum provides a detectable or significant scent to the composition(s).

As noted above, compositions described herein can comprise one or more compounds or mixtures of compounds, e.g., mixtures of compounds added as a single ingredient, which are characterizable as emollient(s). In certain aspects, compositions of the invention can comprise any one or more emollient(s) suitable for dermatological applications known in the art, and which do not detectably or significantly negatively impact the performance of one, some, most, generally all, or all other components/ingredients of the compositions described herein. In certain aspects, the presence of one or more emollient(s), measurably increases the softness, smoothness, or moisture of the skin receiving the composition over the same composition without the one or more emollient(s). In some embodiments, the presence of one or more emollient(s) increases the softness, smoothness, or moisture of the skin receiving the composition over the same composition without the one or more emollient(s) by, for example, at least by about 0.01%, at least by 0.05%, at least by about 0.1%, at least about 0.5%, at least about 1%, at least about 2%, at least about 3%, at least about 5%, or at least about 7%, such as at least about 10%, as measured by an art-accepted method.

As noted above, compositions described herein can comprise one or more compounds or mixtures of compounds, e.g., mixtures of compounds added as a single ingredient, which are characterizable as surfactant(s). In certain aspects, compositions of the invention can comprise any one or more surfactant(s) suitable for dermatological applications known in the art, and which do not detectably or significantly negatively impact the performance of one, some, most, generally all, or all other components/ingredients of the compositions described herein. In certain aspects, the presence of one or more surfactant(s) measurably decreases the surface tension of one or more liquids of the composition over the same composition without the one or more surfactant(s). In some embodiments, the presence of one or more surfactant(s) decreases the surface tension of one or more liquids of the composition over the same composition without the one or more surfactant(s) by, for example, at least by about 0.01%, at least by 0.05%, at least by about 0.1%, at least about 0.5%, at least about 1%, at least about 2%, at least about 3%, at least about 5%, or at least about 7%, such as at least about 10%, as measured by an accepted technique in the art.

As noted above, compositions described herein can comprise one or more compounds or mixtures of compounds, e.g., mixtures of compounds added as a single ingredient, which are characterizable as astringent(s). In certain aspects, compositions of the invention can comprise any one or more astringent(s) suitable for dermatological applications known in the art, and which do not detectably, or significantly negatively impact the performance of one, some, most, generally all, or all other components/ingredients of the compositions described herein. In certain aspects, the presence of one or more astringent(s), measurably increases the contraction of skin cells receiving the composition over the same composition without the one or more astringent(s). In some embodiments, the presence of one or more astringent(s) increases the contraction of skin cells receiving the composition over the same composition without the one or more astringent(s) by, for example, at least by about 0.01%, at least by 0.05%, at least by about 0.1%, at least about 0.5%, at least about 1%, at least about 2%, at least about 3%, at least about S%, or at least about 7%, such as at least about 10%, as measured by an accepted technique in the art.

As noted above, compositions described herein can comprise one or more compounds or mixtures of compounds, e.g., mixtures of compounds added as a single ingredient, which are characterizable as oil-/sebum-absorbent(s). In certain aspects, compositions of the invention can comprise any one or more oil-/sebum-absorbent(s) suitable for dermatological applications known in the art, and which do not detectably or significantly negatively impact the performance of one, some, most, generally all, or all other components/ingredients of the compositions described herein. In certain aspects, the presence of one or more oil-/sebum-absorbent(s) measurably increases the oil-/sebum-absorption of the composition over the same composition without the one or more oil-/sebum-absorbent(s). In some embodiments, the presence of one or more oil-/sebum-absorbent(s) increases the oil-/sebum-absorption of the composition over the same composition without the one or more oil-/sebum-absorbent(s) by, for example, at least by about 0.01%, at least by 0.05%, at least by about 0.1%, at least about 0.5%, at least about 1%, at least about 2%, at least about 3%, at least about 5%, or at least about 7%, such as at least about 10%, as measured by an accepted technique in the art.

As noted above, compositions described herein can comprise one or more compounds or mixtures of compounds, e.g., mixtures of compounds added as a single ingredient, which are characterizable as drying agent(s). In certain aspects, compositions of the invention can comprise any one or more drying agent(s) suitable for dermatological applications known in the art, and which do not detectably or significantly negatively impact the performance of one, some, most, generally all, or all other components/ingredients of the compositions described herein. In certain aspects, the presence of one or more drying agents measurably increases the drying capacity of the composition over the same composition without the one or more drying agents. In some embodiments, the presence of one or more drying agents increases the drying capacity of the composition over the same composition without the one or more drying agents by, for example, at least by about 0.01%, at least by 0.05%, at least by about 0.1%, at least about 0.5%, at least about 1%, at least about 2%, at least about 3%, at least about 5%, or at least about 7%, such as at least about 10%, as measured by an accepted technique in the art.

As noted above, compositions described herein can comprise one or more compounds or mixtures of compounds, e.g., mixtures of compounds added as a single ingredient, which are characterizable as texturizer(s). In certain aspects, compositions of the invention can comprise any one or more texturizer(s) suitable for dermatological applications known in the art, and which do not detectably or significantly negatively impact the performance of one, some, most, generally all, or all other components/ingredients of the compositions described herein. In certain aspects, the presence of one or more drying agents measurably increases one or more textural characteristics of the composition over the same composition without the one or more texturizer(s). In some embodiments, the presence of one or more texturizer(s) increases one or more textural characteristics of the composition over the same composition without the one or more texturizers by, for example, at least by about 0.01%, at least by 0.05%, at least by about 0.1%, at least about 0.5%, at least about 1%, at least about 2%, at least about 3%, at least about 5%, or at least about 7%, such as at least about 10%, as measured by an accepted technique in the art.

As noted above, compositions described herein can comprise one or more compounds or mixtures of compounds, e.g., mixtures of compounds added as a single ingredient, which are characterizable as thickener(s)/viscosity enhancer(s). In certain aspects, compositions of the invention can comprise any one or more thickener(s)/viscosity enhancer(s) suitable for dermatological applications known in the art, and which do not detectably or significantly negatively impact the performance of one, most generally all, or all other components/ingredients of the compositions described herein. In certain aspects, the presence of one or more thickener(s)/viscosity enhancer(s) measurably increases the viscosity of the composition over the same composition without the one or more thickener(s)/viscosity enhancer(s). In some embodiments, the presence of one or more thickener(s)/viscosity enhancers increases the viscosity of the composition over the same composition without the one or more thickener(s)/viscosity enhancer(s) by, for example, at least by about 0.01%, at least by 0.05%, at least by about 0.1%, at least about 0.5° A), at least about 1%, at least about 2%, at least about 3%, at least about 5%, or at least about 7%, such as at least about 10%, as measured by an accepted technique in the art.

As noted above, compositions described herein can comprise one or more compounds or mixtures of compounds, e.g., mixtures of compounds added as a single ingredient, which are characterizable as binding agent(s). In certain aspects, compositions of the invention can comprise any one or more binding agent(s) suitable for dermatological applications known in the art, and which do not detectably or significantly negatively impact the performance of one, some, most, generally all, or all other components/ingredients of the compositions described herein. In certain aspects, the presence of one or more binding agent(s) measurably increases the binding capacity of the composition over the same composition without the one or more binding agent(s). In some embodiments, the presence of one or more binding agent(s) increases the binding capacity of the composition over the same composition without the one or more binding agent(s) by, for example, at least by about 0.01%, at least by 0.05%, at least by about 0.1%, at least about 0.5%, at least about 1%, at least about 2%, at least about 3%, at least about 5%, or at least about 7%, such as at least about 10%, as measured by an accepted technique in the art.

As noted above, compositions described herein can comprise one or more compounds or mixtures of compounds, e.g., mixtures of compounds added as a single ingredient, which are characterizable as chelating agent(s). In certain aspects, compositions of the invention can comprise any one or more chelating agent(s) suitable for dermatological applications known in the art, and which do not detectably or significantly negatively impact the performance of one, some, most, generally all, or all other components/ingredients of the compositions described herein. In certain aspects, the presence of one or more chelating agent(s), measurably increases the metal chelating capacity of the composition over the same composition without the one or more chelating agent(s). In some embodiments, the presence of one or more chelating agent(s) increases the metal chelating capacity of the composition over the same composition without the one or more chelating agent(s) by, for example, at least by about 0.01%, at least by 0.05%, at least by about 0.1%, at least about 0.5%, at least about 1%, at least about 2%, at least about 3%, at least about 5%, or at least about 7%, such as at least about 10%, as measured by an accepted technique in the art.

As noted above, compositions described herein can comprise one or more compounds or mixtures of compounds, e.g., mixtures of compounds added as a single ingredient, which are characterizable as conditioner(s). In certain aspects, compositions of the invention can comprise any one or more conditioner(s) suitable for dermatological applications known in the art, and which do not detectably or significantly negatively impact the performance of one, some, most, generally all, or all other components/ingredients of the compositions described herein. In certain aspects, the presence of one or more conditioner(s) measurably increases the skin conditioning capacity of the composition over the same composition without the one or more conditioner(s). In some embodiments, the presence of one or more conditioner(s) increases the skin conditioning capacity of the composition over the same composition without the one or more conditioner(s) by, for example, at least by about 0.01%, at least by 0.05%, at least by about 0.1%, at least about 0.5%, at least about 1%, at least about 2%, at least about 3%, at least about 5%, or at least about 7%, such as at least about 10%, as measured by an accepted technique in the art.

As noted above, compositions described herein can comprise one or more compounds or mixtures of compounds, e.g., mixtures of compounds added as a single ingredient, which are characterizable as moisturizer(s). In certain aspects, compositions of the invention can comprise any one or more moisturizer(s) suitable for dermatological applications known in the art, and which do not detectably or significantly negatively impact the performance of one, some, most, generally all, or all other components/ingredients of the compositions described herein. In certain aspects, the presence of one or more moisturizer(s) measurably increases the moisturizing capacity of the composition over the same composition without the one or more moisturizer(s). In some embodiments, the presence of one or more moisturizer(s) increases the skin moisturizing capacity of the composition over the same composition without the one or more moisturizer(s) by, for example, at least by about 0.01%, at least by 0.05%, at least by about 0.1%, at least about 0.5%, at least about 1%, at least about 2%, at least about 3%, at least about 5%, or at least about 7%, such as at least about 10%, as measured by an accepted technique in the art.

As noted above, compositions described herein can comprise one or more compounds or mixtures of compounds, e.g., mixtures of compounds added as a single ingredient, which are characterizable as humectant(s). In certain aspects, compositions of the invention can comprise any one or more humectant(s) suitable for dermatological applications known in the art, and which do not detectably or significantly negatively impact the performance of one, some, most, generally all, or all other components/ingredients of the compositions described herein. In certain aspects, the presence of one or more humectant(s) measurably increases the skin's retention or preservation of moisture over the same composition without the one or more humectant(s). In some embodiments, the presence of one or more humectant(s) increases the skin's retention or preservation of moisture over the same composition without the one or more humectant(s) by, for example, at least by about 0.01%, at least by 0.05%, at least by about 0.1%, at least about 0.5%, at least about 1%, at least about 2%, at least about 3%, at least about 5%, or at least about 7%, such as at least about 10%, as measured by an accepted technique in the art.

In certain aspects, compositions of the invention can comprise any one or more compounds or mixtures of compounds providing any two or more, any three or more, any four or more, any 5 or more, any 6 or more, any 7 or more, any 8 or more, any 9 or more, any 10, or more, any 11 or more, or all 12 of the functions provided above, e.g., providing measurable, detectable, or significant emollifying, surfactant, astringent, oil-/sebum-absorbing, drying, texturizing, thickening/viscosity-enhancing, binding, chelating, conditioning, moisturizing, and humectant functions, as measured by art accepted method(s).

As noted above, compositions described herein can comprise one or more compounds or mixtures of compounds, e.g., mixtures of compounds added as a single ingredient, which are characterizable as anti-pruritic (anti-itch) agent(s). In certain aspects, compositions described herein can measurably reduce itching over the same composition lacking the one or anti-pruritic agent(s). In some aspects, the presence of one or more anti-pruritic agent(s) can reduce itching over the same composition without the one or more anti-pruritic agents by for example, at least about 0.01%, at least by 0.05%, at least by about 0.1%, at least about 0.5%, at least about 1%, at least about 2%, at least about 3%, at least about 5%, or at least about 7%, such as at least about 10%, as measured by an accepted technique in the art.

As noted above, compositions described herein can comprise one or more compounds or mixtures of compounds, e.g., mixtures of compounds added as a single ingredient, which are characterizable as stabilizing agent(s). In some aspects, the presence of one or more such stabilizing agent(s) can reduce detectable or significant changes in composition pH, consistency, texture, smell/odor, or appearance from the composition pH, consistency, texture, smell/odor, or appearance at the time of packaging, at the time of opening by the consumer or both, over the same composition without the one or more stabilizing agents by for example, at least about 0.01%, at least by 0.05%, at least by about 0.1%, at least about 0.5%, at least about 1%, at least about 2%, at least about 3%, at least about 5%, or at least about 7%, such as at least about 10%, as measured by an accepted technique in the art.

As noted above, compositions described herein can comprise one or more compounds or mixtures of compounds, e.g., mixtures of compounds added as a single ingredient, which are characterizable as emulsifier(s). In some aspects, the presence of one or more such emulsifying agent(s) can detectably or significantly extend the amount of time the composition remains uniform in consistency (e.g. components do not separate from one another causing, e.g., layering); detectably or significantly maintain the composition in a form identifiable as an emulsion over a period of time under typical conditions, e.g., over the course of the shelf life of the composition, such as, e.g., the time between packaging and complete product use or between the time of packaging and a provided product expiration date when stored under suggested storage conditions or under standard cosmetic storage conditions such as in a dry location at about room temperature; maintain some, most, substantially all, or all of the material in a stable emulsion, such as at least about 80%, at least about 85%, at least about 90%, at least about 95%, at least about 97%, or at least about 99% of the material is maintained in a stable emulsion; or any or all thereof, over the same composition without the one or more emulsifying agent(s) by for example, at least about 0.01%, at least by 0.05%, at least by about 0.1%, at least about 0.5%, at least about 1%, at least about 2%, at least about 3%, at least about 5%, or at least about 7%, such as at least about 10%, as measured by an accepted technique in the art.

As noted above, compositions described herein can comprise one or more compounds or mixtures of compounds, e.g., mixtures of compounds added as a single ingredient, which are characterizable as lubricant(s). In some aspects, the presence of one or more such lubrication agent(s) can detectably or significantly improve the smooth skin feel of the product, improve the soft skin feel of the product, or both, over the same composition without the one or more lubrication agent(s) by for example, at least about 0.01%, at least by 0.05%, at least by about 0.1%, at least about 0.5%, at least about 1%, at least about 2%, at least about 3%, at least about 5%, or at least about 7%, such as at least about 10%, as measured by an accepted technique in the art.

As noted above, compositions described herein can comprise one or more compounds or mixtures of compounds, e.g., mixtures of compounds added as a single ingredient, which are characterizable as barrier forming/protection agent(s). In some aspects, the presence of one or more such barrier forming/protection agent(s) can detectably or significantly maintain skin moisture (e.g., prevent drying), reduce the penetration of skin by unwanted materials, such as penetration of skin impurities, or both, over the same composition without the one or more lubrication agent(s) by for example, at least about 0.01%, at least by 0.05%, at least by about 0.1%, at least about 0.5%, at least about 1%, at least about 2%, at least about 3%, at least about 5%, or at least about 7%, such as at least about 10%, as measured by an accepted technique in the art.

In aspects the compositions of the invention provide detectable or significant anti-fungal activity. In certain aspects, compositions of the invention can detectably or significantly prevent or reduce fungal activity (e.g., the number of fungal colonies, reduce the speed of fungal growth, extend the time of the appearance of a fungus, or any or all thereof) over the same composition without the one or more anti-fungal agent(s), over one or more comparable marketed products, e.g., other sulfur-containing dermatological products, or both, by for example, at least about 0.01%, at least by 0.05%, at least by about 0.1%, at least about 0.5%, at least about 1%, at least about 2%, at least about 3%, at least about 5%, or at least about 7%, such as at least about 10%, at least about 15%, at least about 20%, at least about 25%, at least about 30%, at least about 35%, at least about 40%, at least about 45%, or at least about 50% or more, as measured by an accepted technique in the art.

In aspects the compositions of the invention provide detectable or significant anti-yeast activity. In certain aspects, compositions of the invention can detectably or significantly prevent or reduce yeast activity (e.g., the number of yeast colonies, reduce the speed of yeast growth, extend the time of the appearance of a yeast, or any or all thereof) over the same composition without the one or more anti-yeast agent(s), over one or more comparable marketed products, e.g., other sulfur-containing dermatological products, or both, by for example, at least about 0.01%, at least by 0.05%, at least by about 0.1%, at least about 0.5%, at least about 1%, at least about 2%, at least about 3%, at least about 5%, or at least about 7%, such as at least about 10%, at least about 15%, at least about 20%, at least about 25%, at least about 30%, at least about 35%, at least about 40%, at least about 45%, or at least about 50% or more, as measured by an accepted technique in the art.

In aspects the compositions of the invention provide detectable or significant anti-bacterial activity. In certain aspects, compositions of the invention can detectably or significantly prevent or reduce bacterial activity (e.g., the number of bacterial colonies, reduce the speed of bacterial growth, extend the time of the appearance of a bacterial colony, or any or all thereof) over the same composition without the one or more anti-bacterial agent(s), over one or more comparable marketed products, e.g., other sulfur-containing dermatological products, or both, by for example, at least about 0.01%, at least by 0.05%, at least by about 0.1%, at least about 0.5%, at least about 1%, at least about 2%, at least about 3%, at least about 5%, or at least about 7%, such as at least about 10%, at least about 15%, at least about 20%, at least about 25%, at least about 30%, at least about 35%, at least about 40%, at least about 45%, or at least about 50% or more, as measured by an accepted technique in the art.

In aspects the compositions of the invention provide detectable or significant anti-pruritic activity. In certain aspects, compositions of the invention can detectably or significantly prevent or reduce itchiness experienced by the user over the same composition without the one or more anti-pruritic agent(s), over one or more comparable marketed products, e.g., other sulfur-containing dermatological products, or both, by for example, at least about 0.01%, at least by 0.05%, at least by about 0.1%, at least about 0.5%, at least about 1%, at least about 2%, at least about 3%, at least about 5%, or at least about 7%, such as at least about 10%, at least about 15%, at least about 20%, at least about 25%, at least about 30%, at least about 35%, at least about 40%, at least about 45%, or at least about 50% or more, as measured by an accepted technique in the art.

In aspects the compositions of the invention provide detectable or significant anti-inflammatory activity. In certain facets, compositions of the invention can detectably or significantly prevent or reduce skin inflammation experienced by the user over the same composition without the one or more anti-inflammatory agent(s), over one or more comparable marketed products, e.g., other sulfur-containing dermatological products, or both, by for example, at least about 0.01%, at least by 0.05%, at least by about 0.1%, at least about 0.5%, at least about 1%, at least about 2%, at least about 3%, at least about 5%, or at least about 7%, such as at least about 10%, at least about 15%, at least about 20%, at least about 25%, at least about 30%, at least about 35%, at least about 40%, at least about 45%, or at least about 50% or more, as measured by an accepted technique in the art.

In some aspects, the compositions described herein are capable of providing each of detectable or significant anti-fungal activity, anti-yeast activity, anti-bacterial activity, anti-pruritic activity, and anti-inflammatory activity as described above.

In certain aspects, the invention describes compositions suitable for topical application to the skin of a mammal, e.g., a human, comprising sulfur as the sole active and further comprising long carbon chain compounds, such as long chain fatty acid compounds and one or more other functional compounds, and whereby the composition(s) is suitable for the treatment of acne. In some facets, the composition carries the FDA monograph for acne. According to some aspects, compositions described herein can detectably or significantly reduce symptoms of acne such as papules, pustules (pimples), redness, scarring, whiteheads (closed plugged pores), blackheads (open plugged pores), nodules, skin bump crusting, and cysts, or any combination thereof. In certain facets, compositions described herein can reduce one or more symptoms of acne, e.g., any one or more visual appearance characteristics of acne, by at least about 0.5%, at least about 1%, at least about 1.5%, at least about 2%, at least about 2.5%, at least about 3%, at least about 3.5%, at least about 4%, at least about 4.5%, or at least about 5%, such as at least approximately 5.5%, at least approximately 6%, at least approximately 6.5%, at least approximately 7%, at least approximately 7.5%, at least approximately 8%, at least approximately 8.5%, at least approximately 9%, at least approximately 9.5%, or at least approximately 10% or even more, such as at least about 15%, at least about 20%, at least about 25%, at least about 30%, at least about 35%, at least about 40%, at least about 45%, at least about 50%, or even more, when applied once per day or twice per day, within approximately 1 month of first use, such as within approximately 3 weeks, e.g., within approximately 2 weeks, within approximately 10 days, within approximately 7 days, within approximately 6 days, within approximately 5 days, within approximately 4 days, within approximately 3 days, within approximately 2 days, or within approximately 24 hours of first use.

In certain aspects, the invention describes compositions suitable for topical application to the skin of a mammal, e.g., a human, comprising sulfur as the sole active and further comprising long carbon chain compounds, such as long chain fatty acid compounds and one or more other functional compounds whereby the composition(s) is suitable for the treatment of pimples. In certain facets, compositions described herein can reduce one or more of the size, number, redness or visibility, or speed of spread of pimple(s), by at least about 0.5%, at least about 1%, at least about 1.5%, at least about 2%, at least about 2.5%, at least about 3%, at least about 3.5%, at least about 4%, at least about 4.5%, or at least about 5%, such as at least approximately 5.5%, at least approximately 6%, at least approximately 6.5%, at least approximately 7%, at least approximately 7.5%, at least approximately 8%, at least approximately 8.5%, at least approximately 9%, at least approximately 9.5%, or at least approximately 10% or even more, such as at least about 15%, at least about 20%, at least about 25%, at least about 30%, at least about 35%, at least about 40%, at least about 45%, at least about 50%, or even more, when applied once per day or twice per day, within approximately 1 month of first use, such as within approximately 3 weeks, e.g., within approximately 2 weeks, within approximately 10 days, within approximately 7 days, within approximately 6 days, within approximately 5 days, within approximately 4 days, within approximately 3 days, within approximately 2 days, or within approximately 24 hours of first use.

In certain aspects, the invention describes compositions suitable for topical application to the skin of a mammal, e.g., a human, comprising sulfur as the sole active and further comprising long carbon chain compounds, such as long chain fatty acid compounds and one or more other functional compounds whereby the composition(s) is suitable for the treatment of rosacea. In certain facets, compositions described herein can reduce one or more of the symptoms of rosacea such as, e.g., any one or more of erythema, telangiectasia, or both, or conditions such as inflammatory lesions, burning, pruritus, scaling, swelling (edema), or combinations thereof, by a detectable or significant amount, such as by at least about 0.5%, at least about 1%, at least about 1.5%, at least about 2%, at least about 2.5%, at least about 3%, at least about 3.5%, at least about 4%, at least about 4.5%, or at least about 5%, such as at least approximately 5.5%, at least approximately 6%, at least approximately 6.5%, at least approximately 7%, at least approximately 7.5%, at least approximately 8%, at least approximately 8.5%, at least approximately 9%, at least approximately 9.5%, or at least approximately 10% or even more, such as at least about 15%, at least about 20%, at least about 25%, at least about 30%, at least about 35%, at least about 40%, at least about 45%, at least about 50%, or even more, when applied once per day or twice per day, within approximately 1 month of first use, such as within approximately 3 weeks, e.g., within approximately 2 weeks, within approximately 10 days, within approximately 7 days, within approximately 6 days, within approximately 5 days, within approximately 4 days, within approximately 3 days, within approximately 2 days, or within about 24 hours of first use.

In certain aspects, the invention describes compositions suitable for topical application to the skin of a mammal, e.g., a human, comprising sulfur as the sole active and further comprising long carbon chain compounds, such as long chain fatty acid compounds and one or more other functional compounds whereby the composition(s) is suitable for the treatment of seborrheic dermatitis (dandruff). In certain facets, compositions described herein can reduce one or more of the symptoms of seborrheic dermatitis such as, e.g., any one or more of dandruff, skin scaling, or both, or any other known symptoms thereof, in a detectable or significant amount, such as by at least about 0.5%, at least about 1%, at least about 1.5%, at least about 2%,, at least about 2.5%, at least about 3%, at least about 3.5%, at least about 4%, at least about 4.5%, or at least about 5%, such as at least approximately 5.5%, at least approximately 6%, at least approximately 6.5%, at least approximately 7%, at least approximately 7.5%, at least approximately 8%, at least approximately 8.5%, at least approximately 9%, at least approximately 9.5%, or at least approximately 10% or even more, such as at least about 15%, at least about 20%, at least about 25%, at least about 30%, at least about 35%, at least about 40%, at least about 45%, at least about 50%, or even more, when applied once per day or twice per day, within approximately 1 month of first use, such as within approximately 3 weeks, e.g., within approximately 2 weeks, within approximately 10 days, within approximately 7 days, within approximately 6 days, within approximately 5 days, within approximately 4 days, within approximately 3 days, within approximately 2 days, or within approximately 24 hours of first use.

In certain aspects, the invention describes compositions suitable for topical application to the skin of a mammal, e.g., a human, comprising sulfur as the sole active and further comprising long carbon chain compounds, such as long chain fatty acid compounds and one or more other functional compounds whereby the composition(s) is suitable for the treatment of perioral dermatitis. In certain facets, compositions described herein can reduce one or more of the symptoms of perioral dermatitis such as, e.g., any one or more of rash, pustules, scaling, or combination thereof, in a detectable or significant amount, such as by at least about 0.5%, at least about 1%, at least about 1.5%, at least about 2%, at least about 2.5%, at least about 3%, at least about 3.5%, at least about 4%, at least about 4.5%, or at least about 5%, such as at least approximately 5.5%, at least approximately 6%, at least approximately 6.5%, at least approximately 7%, at least approximately 7.5%, at least approximately 8%, at least approximately 8.5%, at least approximately 9%, at least approximately 9.5%, or at least approximately 10% or even more, such as at least about 15%, at least about 20%, at least about 25%, at least about 30%, at least about 35%, at least about 40%, at least about 45%, at least about 50%, or even more, when applied once per day or twice per day, within approximately 1 month of first use, such as within approximately 3 weeks, e.g., within approximately 2 weeks, within approximately 10 days, within approximately 7 days, within approximately 6 days, within approximately 5 days, within approximately 4 days, within approximately 3 days, within approximately 2 days, or within about 24 hours of first use.

In certain aspects, the invention describes compositions suitable for topical application to the skin of a mammal, e.g., a human, comprising sulfur as the sole active and further comprising long carbon chain compounds, such as long chain fatty acid compounds and one or more other functional compounds whereby the composition(s) is suitable for the treatment of tinea versicolor. In certain facets, compositions described herein can reduce one or more of the symptoms of tinea versicolor such as, e.g., any one or more of skin discoloration, scaling, itching, or combination thereof, or other symptoms known to be associated with this disease, in a significant or detectable amount, such as by at least about 0.5%, at least about 1%, at least about 1.5%, at least about 2%, at least about 2.5%, at least about 3%, at least about 3.5%, at least about 4%, at least about 4.5%, or at least about 5%, such as at least approximately 5.5%, at least approximately 6%, at least approximately 6.5%, at least approximately 7%, at least approximately 7.5%, at least approximately 8%, at least approximately 8.5%, at least approximately 9%, at least approximately 9.5%, or at least approximately 10% or even more, such as at least about 15%, at least about 20%, at least about 25%, at least about 30%, at least about 35%, at least about 40%, at least about 45%, at least about 50%, or even more, when applied once per day or twice per day, within approximately 1 month of first use, such as within approximately 3 weeks, e.g., within approximately 2 weeks, within approximately 10 days, within approximately 7 days, within approximately 6 days, within approximately 5 days, within approximately 4 days, within approximately 3 days, within approximately 2 days, or within approximately 24 hours of first use.

In certain aspects, the invention describes compositions suitable for topical application to the skin of a mammal, e.g., a human, comprising sulfur as the sole active and further comprising long carbon chain compounds, such as long chain fatty acid compounds and one or more other functional compounds whereby the composition(s) is suitable for the treatment of pityrosporon folliculitis. In certain facets, compositions described herein can reduce one or more of the symptoms of pityrosporon folliculitis such as, e.g., any one or more of itching, pustules, or both, or any other symptom of this disease, in a significant or detectable amount, such as by at least about 0.5%, at least about 1%, at least about 1.5%, at least about 2%, at least about 2.5%, at least about 3%, at least about 3.5%, at least about 4%, at least about 4.5%, or at least about 5%, such as at least approximately 5.5%, at least approximately 6%, at least approximately 6.5%, at least approximately 7%, at least approximately 7.5%, at least approximately 8%, at least approximately 8.5%, at least approximately 9%, at least approximately 9.5%, or at least approximately 10% or even more, such as at least about 15%, at least about 20%, at least about 25%, at least about 30%, at least about 35%, at least about 40%, at least about 45%, at least about 50%, or even more, when applied once per day or twice per day, within approximately 1 month of first use, such as within approximately 3 weeks, e.g., within approximately 2 weeks, within approximately 10 days, within approximately 7 days, within approximately 6 days, within approximately 5 days, within approximately 4 days, within approximately 3 days, within approximately 2 days, or within about 24 hours of first use.

In certain aspects, the invention describes compositions suitable for topical application to the skin of a mammal, e.g., a human, comprising sulfur as the sole active and further comprising long carbon chain compounds, such as long chain fatty acid compounds and one or more other functional compounds whereby the composition(s) is suitable for the treatment of mild dermatitis such as that which is caused by insect bite(s). In certain facets, compositions described herein can reduce one or more of the symptoms of mild dermatitis such as, e.g., any one or more of itching, redness, swelling, e.g., swelling associated with an insect bite, or rash, scaling, or other symptom of dermatitis, by a significant or detectable amount, such as by at least about 0.5%, at least about 1%, at least about 1.5%, at least about 2%, at least about 2.5%, at least about 3%, at least about 3.5%, at least about 4%, at least about 4.5%, or at least about 5%, such as at least approximately 5.5%, at least approximately 6%, at least approximately 6.5%, at least approximately 7%, at least approximately 7.5%, at least approximately 8%, at least approximately 8.5%, at least approximately 9%, at least approximately 9.5%, or at least approximately 10% or even more, such as at least about 15%, at least about 20%, at least about 25%, at least about 30%, at least about 35%, at least about 40%, at least about 45%, at least about 50%, or even more, when applied once per day or twice per day, within approximately 1 month of first use, such as within approximately 3 weeks, e.g., within approximately 2 weeks, within approximately 10 days, within approximately 7 days, within approximately 6 days, within approximately 5 days, within approximately 4 days, within approximately 3 days, within approximately 2 days, or within approximately 24 hours of first use.

In certain facets, the compositions comprise the anti-pruritic Calamine or the ingredients or functionally equivalent ingredients to Calamine such that the invention describes compositions capable of treating any one or more of acne, pimples, rosacea, seborrheic dermatitis, perioral dermatitis, tinea versicolor, pityrosporon folliculitis, and mild dermatitis while concurrently being capable of treating mild itchiness from any of such conditions or from, e.g., sunburn, insect bite(s), poison ivy, or other mild skin conditions. In certain facets, compositions described herein can reduce the mild itchiness from any of such conditions or from, e.g., sunburn, insict bite(s), poison ivy, or other mild skin condition(s) by at least about 0.5%, at least about 1%, at least about 1.5%, at least about 2%, at least about 2.5%, at least about 3%, at least about 3.5%, at least about 4%, at least about 4.5%, or at least about 5%, such as at least approximately 5.5%, at least approximately 6%, at least approximately 6.5%, at least approximately 7%, at least approximately 7.5%, at least approximately 8%, at least approximately 8.5%, at least approximately 9%, at least approximately 9.5%, or at least approximately 10% or even more, such as at least about 15%, at least about 20%, at least about 25%, at least about 30%, at least about 35%, at least about 40%, at least about 45%, at least about 50%, or even more, when applied once per day or twice per day, within approximately 1 month of first use, such as within approximately 3 weeks, e.g., within approximately 2 weeks, within approximately 10 days, within approximately 7 days, within approximately 6 days, within approximately 5 days, within approximately 4 days, within approximately 3 days, within approximately 2 days, or within about 24 hours of first use.

In certain facets, the compositions of the invention are capable of treating such conditions such as acne, pimples, rosacea, seborrheic dermatitis, perioral dermatitis, tinea versicolor, pityrosporon folliculitis, and mild dermatitis without causing detectable or significant scabbing of the underlying skin.

According to some embodiments, compositions of the invention comprise at least 10% sulfur and use of the compositions of the invention caused no detectable or significant side effects of sulfur commonly known in the art as reported by at least 50% of people using the composition(s). In certain aspects, compositions of the invention comprise at least 10% sulfur and at least 50% of people using the composition report no detectable or significant skin burning, tingling, stinging, itching, redness, peeling, dryness, excessive oil, or any combination thereof. In certain aspects, at least 50% of people using the composition(s) report a detectably or significantly reduced frequency or severity of any one or more of skin burning, tingling, stinging, itching, redness, peeling, dryness, or excessive oil, or any combination thereof, compared to one or more other topical dermatological products comprising at least 10% sulfur.

In certain respects, the invention provides topical compositions comprising sulfur as the sole active and further comprising long carbon chain compounds, such as long chain fatty acid compounds and one or more other functional compounds ingredient which are non-comedogenic. In some facets, the invention provides topical compositions comprising sulfur as the sole active and further comprising long carbon chain compounds, such as long chain fatty acid compounds and one or more other functional compounds agent comprising detectable or significant anti-fungal activity, anti-yeast activity, anti-bacterial activity, anti-pruritic activity, or anti-inflammatory activity while providing cosmetically desirable characteristics such as having in aspects mattifying properties, non-tackiness, and being determined to have good skin feel or cosmetic elegance as reported by a significant number of users of the product, such as by at least 50% of users in an appropriately conducted survey or study.

In certain facets, the invention describes compositions comprising sulfur in the form of colloidal sulfur, and further comprising long chain carbon compounds such as long carbon chain fatty acids (e.g., fatty acids having at least 17 carbons) provided in a formulation suitable for topical application to the skin of a mammal, the colloidal sulfur present in the composition along with one or more of a compound or mixture of two or more compounds characterizable as emollient(s), surfactant(s), astringent(s), oil-, e.g., sebum-absorbent(s), drying agent(s), texturizer(s), thickener(s)/viscosity enhancer(s), binding agent(s), conditioner(s), moisturizer(s), humectant(s), anti-pruritic (anti-itch) agent(s), stabilizing agent(s), emulsifier(s), lubricant(s), or barrier-forming or protection agent(s), whereby the composition provides detectable or significant anti-fungal activity, anti-yeast activity, anti-bacterial activity, anti-pruritic activity, and anti-inflammatory activity, and further whereby the composition(s) is suitable for the treatment of any one or more of acne, pimples, rosacea, seborrheic dermatitis (dandruff), perioral dermatitis, tinea versicolor, pityrosporon folliculitis, and mild dermatitis.

According to some aspects, the invention provides methods of treating any one or more of acne, pimples, rosacea, seborrheic dermatitis (dandruff), perioral dermatitis, tinea versicolor, pityrosporon folliculitis, and mild dermatitis in a human subject using any composition(s) provided here.

According to some aspects, the invention provides methods of using any one or more of the compositions described herein to treat a subject suffering from one or more symptoms of, or having been diagnosed by a medical professional with, any one or more of acne, pimples, rosacea, seborrheic dermatitis (dandruff), perioral dermatitis, tinea versicolor, pityrosporon folliculitis, and mild dermatitis.

According to some aspects, the invention provides methods of manufacturing any one or more of the compositions described herein.

In certain aspects, the compositions of the invention are characterizable by such compounds which are not present. In aspects, the topical compositions of the present invention are non-foaming. In certain embodiments, the topical compositions of the present invention do not comprise any one or more of a plasticizer(s), hydrolyzed protein(s), or a histamine antagonist.

### EXAMPLE

Table 1 below provides an exemplary composition of the invention comprising a combination of a high sulfur concentration colloidal sulfur composition ingredient, long chain fatty acid components, and other functional ingredients, such as a silicone and squalane. This Example is presented to illustrate how the various ingredients described herein can be combined to generate a formulation having some or all of the beneficial properties described above in connection with formulations of the invention. While this Example reflects an aspect of the invention, it should not, however, be used to limit the scope of the invention in any way.

| | | % WT/ |
|---|---|---|
| DESCRIPTION | INCI | WT |
| Deionized Water | Water (aqua) | 54.33 |
| Dissolvine ® NA2-P | Disodium EDTA | 0.05 |
| Glycerine 99.7% USP | Glycerin, water (aqua) | 3 |
| Sepinov ™ EMT 10 | Hydroxyethyl | 1 |
| | acrylate/sodiurn | |
| | acryloyldimethyl taurate | |
| | copolymer | |
| Cetiola CC | Dicaprylyl Carbonate | 4 |
| Promulgen ™ D | Cetearyl alcohol, | 2 |
| | Ceteareth-20 | |
| Jarplex ™ SB-WD | Shea butter glycerides | 2 |
| Stearic acid | Stearic acid | 2 |
| Sunflower oil, organic, | Helianthus Annus | 5 |
| high oleic | (Sunflower) seed oil | |
| Simulsol ™ 165 | PEG-100 stearate, | 2.5 |
| | glyceryl stearate | |
| Dimethisil® DM-350 | Dimethicone | 2 |
| Neossance ™ squalane | Squalane | 3 |
| Vitamin E acetate | Tocopheryl acetate | 0.3 |
| Premier Magnolia Super | Magnolia Officinalis Bark | 0.1 |
| Extract | Extract | |
| Calamine, Powder, USP | Calamine | 2 |
| Sulfidal® | Sulfur, Acacia Senegal | 13.35 |
| | Gum | |
| Botanistat PF-64 | Phenoxyethanol, | 1 |
| | caprylyl glycol, hexylene | |
| | glycol, | |
| | ethylhexylglycerin | |
| SymCalmin® 143535 | Penthylene glycol, | 2 |
| | butylene glycol, | |
| | hydroxyphenyl | |
| | propamidobenzoid acid | |
| Bisabolol | Bisabolol | 0.2 |
| Tea Tree Oil #448700 | Melaleuca Arternifolia | 0.04 |
| | (Tea Tree) leaf oil | |
| Fragrance Apple of My | Fragrance (Parfum) | 0.13 |
| Eye UAC08799/00 | | |

What is claimed is:

1. A dermatologically suitable topical and aqueous formulation comprising (a) at least 50% water, (b) at least about 12% of a colloidal sulfur ingredient that is at least about 66% composed of sulfur, (c) about 3.5-7% of an oil comprising about 60-90% C18 fatty acids, (d) about 3-5% dicaprylyl carbonate, (d) at least about 3-6% of one or more additional C18 fatty acid compounds, (e) about 1.5-3% of a silicone, (f) about 2-4% squalane, and (g) about 1.5-3% calamine.

2. The formulation of claim 1, comprising about 5% sunflower oil.

3. The formulation of claim 1 or claim 2, comprising about 1.75-2.25% dimethicone.

4. The formulation of any one of claims 1-3, further comprising about 0.5-1.5% Hydroxyethyl Acrylate/Sodium Acryloyldimethyl Taurate Copolymer.

5. The formulation of any one of claims 1-4, further comprising about 0.5%-1.5% of a mixture of Phenoxyethanol, Caprylyl Glycol, Hexylene Glycol, and Ethylhexylglycerin.

6. The formulation of any one of claims 1-5, further comprising about 1.5%-3% Shea Butter Glycerides.

7. The formulation of any one of claims 1-6, further comprising about 0.03-0.07% disodium EDTA; about 0.15-0.25% of a terpene, such as bisabolol; or both.

8. The formulation of any one of claims 1-7, wherein the sulfur is the primary or only active pharmaceutical ingredient in the formulation.

9. A method of treating a skin condition in a patient comprising administering an effective amount of a composition according to any one of claims 1-8 to the patient for a sufficient period to detectably or significantly reduce one or more symptoms of the skin condition.

10. The method of claim 9, wherein the skin condition comprises acne.
